(19) United States
(12) Patent Application Publication
(10) Pub. No.: US 2005/0246023 A1
(43) Pub. Date: Nov. 3, 2005

(54) Disc shunt for treating back pain

(76) Inventor: Jeffrey E. Yeung, San Jose, CA (US)

Correspondence Address: Jeffrey E. Yeung, 834 North White Road, San Jose, CA, 95127-1024, US

(21) Appl. No.: 11/165,076

(22) Filed: Jun. 22, 2005

Related U.S. Application Data
(63) Continuation-in-part of application No. 10/840,816, filed on May 7, 2004.
(63) Continuation-in-part of application No. 10/470,181, filed on Jul. 21, 2003.
(60) Provisional application No. 60/582,228, filed on Jun. 22, 2004.
(60) Provisional application No. 60/587,837, filed on Jul. 14, 2004.
(60) Provisional application No. 60/660,120, filed on Mar. 8, 2005.
(60) Provisional application No. 60/268,666, filed on Feb. 13, 2001.
(60) Provisional application No. 60/297,556, filed on Jun. 11, 2001.
(60) Provisional application No. 60/310,131, filed on Aug. 3, 2001.
(60) Provisional application No. 60/325,111, filed on Sep. 26, 2001.
(60) Provisional application No. 60/330,260, filed on Oct. 17, 2001.

(51) Int. Cl. A61F 2/44

(52) U.S. Cl. 623/17.11; 604/8

(57) ABSTRACT

The intervertebral disc is avascular. With aging, nutrients and oxygen transporting through the endplates diminish. The disc degenerates, and pain ensues. Conduits are delivered through a pedicle or vertebral body into the intervertebral disc to re-establish the exchange of nutrients and waste between the disc and bodily circulation to slow, stop or reverse disc degeneration and relieve pain. Endplate plugs may be deployed to seal gaps between the conduits and the endplates to prevent immune responses to the nucleus pulposus and to preserve the hydrostatic pressure within the disc.

### CROSS-REFERENCES TO OTHER APPLICATIONS

This application is a continuation-in-part of U.S. patent application Ser. No. 10/840,816 filed on May 7, 2004. This application also claims priority of U.S. Provisional Applications 60/582,228 filed on Jun. 22, 2004; 60/587,837 filed on Jul. 14, 2004; and 60/660,120 filed on Mar. 8, 2005.

This application is also a continuation-in-part of U.S. patent application Ser. No. 10/470,181, filed on Jul. 21, 2003, which is a National Stage Application of PCT/US02/04301 filed Feb. 13, 2002, which claimed priority of U.S. Provisional Applications 60/268,666 filed on Feb. 13, 2001; 60/297,556 filed on Jun. 11, 2001; 60/310,131 filed on Aug. 3, 2001; 60/325,111 filed on Sep. 26, 2001; and 60/330,260 filed on Oct. 17, 2001. This application also claims priority of U.S. Provisional Applications 60/468,770 filed on May 7, 2003; 60/480,057 filed on Jun. 20, 2003; 60/503,553 filed on Sep. 16, 2003; and 60/529,065 filed on Dec. 12, 2003.

### FIELD OF INVENTION

This invention relates to devices and methods to deliver and seal a disc shunt to re-establish the transport of nutrients and waste between the disc and vertebral body to halt, decrease or reverse disc degeneration. As a result, back pain is reduced or alleviated.

### BACKGROUND

The intervertebral disc absorbs most of the compressive load of the spine with the facet joints of the vertebral bodies sharing approximately 16%. The disc consists of three distinct parts: the nucleus pulposus, the annular layers and the cartilaginous endplates. The disc maintains its structural properties largely through its ability to attract and retain water. A normal disc contains 80% water in the nucleus pulposus. The nucleus pulposus within a normal disc is rich in water retaining sulfated glycosaminoglycans, which create the swelling pressure necessary to provide tensile stress within the collagen fibers of the annulus. The swelling pressure produced by high water content is crucial to support the annular layers and sustain compressive loads.

In adults, the intervertebral disc is avascular. Survival of the disc cells depends on nutrients supplied from external blood vessels. Penetration of nutrients and oxygen into the disc can be diffusion or pressure driven. Diffusion of nutrients flows from high to low concentration. Nutrients also flow from high to low pressure area. The sources of nutrients and oxygen are from (1) peripheral blood vessels adjacent to the outer annulus, and (2) vertebral body through the endplate into the disc. Diffusion of nutrients from peripheral blood vessels can only reach up to 1 cm into the annular layers of the disc. However, an adult disc can be as large as 5 cm in diameter, leaving the inner disc inadequately supplied with nutrients from the peripheral blood vessels. Hence permeation of nutrients and oxygen through cranial and caudal cartilaginous endplates is crucial for maintaining the health of the nucleus pulposus and inner annular layers of the disc.

Calcium pyrophosphate and hydroxyapatite are commonly found in the endplate and nucleus pulposus. Beginning as young as 18 years of age, calcified layers begin to accumulate in the cartilaginous endplate. The blood vessels and capillaries at the bone-cartilage interface are gradually occluded by the build-up of the calcified layers. When the endplate is obliterated by the calcified layers, nutrient transport through the endplate is greatly hindered. Sulfate is one of the restricted nutrients for biosynthesizing the water-retaining sulfated glycosaminoglycans. As a result, the concentration of sulfated glycosaminoglycans decreases, leading to lower water content and swelling pressure within the nucleus pulposus. During normal daily compressive loading on the spine, the reduced pressure within the nucleus pulposus can no longer distribute the forces evenly along the circumference of the inner annulus to keep the lamellae bulging outward. As a result, the inner lamellae sag inward while the outer annulus continues to bulge outward, causing delamination of the annular layers.

The shear stresses causing annular delamination and bulging are highest at the posteriolateral portions adjacent to the neuroforamen. The nerve is confined within the neuroforamen between the disc and the facet joint. Hence, the nerve at the neuroforamen is vulnerable to impingement by the bulging disc or bone spurs.

The nucleus pulposus is thought to function as “the air in a tire” to pressurize the disc. With inadequate swelling pressure, the degenerated disc exhibits unstable movements, similar to that of a flat tire. Approximately 20-30% of low-back-pain patients have been diagnosed as having spinal segmental instability. The pain may originate from stress and increased load on the facet joints and/or surrounding ligaments.

In addition, the calcified endplate also hinders permeation of oxygen into the disc. Oxygen concentration in the central part of the nucleus is extremely low. Under anaerobic conditions, metabolic production of lactic acid increases, leading to acidic conditions within the disc. Lactic acid diffuses through micro-tears in the annulus and irritates the richly innervated posterior longitudinal ligament, facet joint and/or nerve root. Studies indicate that lumbar pain correlates well with low pH. The mean pH of symptomatic discs was significantly lower than the mean pH of normal discs. Currently, no intervention other than discectomy stops or reduces the production of lactic acid.

Conduits for re-establishing the exchange of nutrients and waste between the degenerative disc and bodily circulation is described in PCT/US2004/14368 (WO 2004/101015), and U.S. application Ser. No. 10/840,816 by J. Yeung and T. Yeung, both applications filed on May 7, 2004. The U.S. Ser. No. 10/840,816 is a continuation-in-part application to U.S. Ser. No. 10/470,181 by J. Yeung and T. Yeung on Jul. 21, 2003 from PCT/US2002/04301 on Feb. 13, 2002 with priorities dated on Feb. 13, 2001, Jun. 11, 2001, Aug. 3, 2001, Sep. 26, 2001 and Oct. 17, 2001. By re-supplying the cells within the disc with nutrients, biosynthesis of sulfated glycosaminoglycans may increase to retain additional water and sustain compressive loading. Hence, segmental instability and excessive loading on facet joints are minimized. With the presence of additional oxygen, production of lactic acid may decrease to minimize acidic irritation. Both retaining additional water and minimizing lactic acid build-up within the disc may halt or reverse disc degeneration and alleviate back pain.

A method providing nutrients to an intervertebral disc through a porous stent or a cannulated element is proposed in U.S. Pat. No. 6,685,695 by Bret Ferree on Feb. 3, 2004. U.S. Pat. No. 6,685,695 and related applications have not mentioned specific method, delivery device or specification of the porous stent or cannulated element. Due to surrounding nerves, shielding of spinal structure and adjacent blood vessels, the method and delivery device for implanting the stent or cannulated element at the endplate are far from obvious. In addition, endplate punctures to provide passages for nutrients entering into the disc have been proposed in PCT/US2002/04301 by J. Yeung and T. Yeung on Feb. 13, 2002 with provisional application filed on Feb. 13, 2001. Furthermore, nucleus content of the disc is immunologic. Large pores in a stent or cannulated element provide sizable entries for IgG, IgM, interleukins-6, prostaglandin E₂, giant cells or other immune responsive component to enter into the disc, which can cause significant immunologic reactions. Through large pores, the nucleus content can also be extruded from the disc and cause immunological response, as seen around herniated discs.

Discs L4-5 and L5-S1 are shielded by the iliac, not accessible by straight needle from outside to deliver the conduit into the disc. However, through the pedicle of the vertebral body, the elastically curved needle proposed in PCT/US2004/14368 (WO 2004/101015) can puncture through the calcified endplate to deliver the conduit for nutrient and lactate exchange.

### SUMMARY OF INVENTION

This invention includes new methods and devices for implanting a conduit and a plug to seal the gap between the conduit and the endplate. Since discs L4-5 and L5-S1 are shielded by the iliac, a method using an elastically curved needle through the pedicle to puncture and deliver the conduit at the endplate is proposed. In addition, another proposed method is to drill through the sacrum into lumbar vertebral bodies to implant a conduit through multiple discs.

In the supine position, the pressure within the shunted disc is low. Nutrients and oxygen from the vertebral body are transported through the conduit into the deprived cells within the, disc. Biosynthesis of sulfated glycosaminoglycans may substantially increase to retain additional water to sustain the compressive load, ease strain on the facet joint and minimize segmental instability. In addition, anaerobic production of lactic acid may decrease with the presence of oxygen. During daily activities, pressure within the shunted disc is high. Lactic acid, carbon dioxide and metabolic waste within the disc are expelled through the conduit into bodily circulation. As a result, metabolic conditions within the shunted disc is normalized. The disc degenerative process is halted or reversed to reduce or alleviate back pain.

### REFERENCE NUMBER

100 Intervertebral disc
101 Needle
105 Endplate
106 Cartilage
108 Calcified layer or blockade
109 Plunger
111 Rectum
112 Blood vessels
117 Endoscope
118 Nerve
119 Colon
120 Inferior fascia pelvic diaphragm
121 Neuroforamen
123 Spinal cord
126 Conduit or shunt
128 Nucleus pulposus
129 Facet joint
136 External anal sphincter muscle
137 Coccyx
138 Anococcygeal body
139 Gluteus maximus muscle
140 Sacrum
141 Blunt obturator
142 Superior articular process
143 Inferior articular process
144 Blunt rod
145 Colon positioner
146 Suction cup
147 Positioner body
148 Positioner handle
149 Vacuum line
150 Drill
151 Genital
152 Puncture site
159 Vertebral body
163 Coating
191 Strain relieving element
194 Nerve root
195 Posterior longitudinal ligament
220 Rigid needle
230 Sheath
269 Lumen of needle
271 Plug sleeve
278 Pedicle
279 Drill stop
290 Cutting groove
292 Endplate plug
293 Plug slit
294 Plug thread
295 Plug lumen
296 Plug nut
297 Drill base
298 Drill grip
299 Drill fastener
300 Drill shaft
301 Gear A
302 Second gear
303 Drive hole
304 Fastening nut
305 Crank handle
306 Drill housing
307 Bolt
308 Nut
309 Needle slit
310 Bevel
311 Slide
312 Slide lumen
313 Drill sleeve
314 Lumen of drill or core
315 Cutting element

### DESCRIPTION OF THE DRAWINGS

FIG. 1 shows a pedicle 278 punctured by a rigid needle 220 carrying an elastically curved needle 101 containing a conduit 126 (not shown) and a plunger 109.

FIG. 2 depicts the superior view of the vertebral body 159 with the rigid needle 220 puncturing through the pedicle 278.

FIG. 3 shows insertion of the rigid needle 220, elastically curved needle 101, conduit 126 and plunger 109 through the pedicle 278 of the vertebral body 159.

FIG. 4 shows deployment of the elastically curved needle 101 from the rigid needle 220, puncturing through the calcified endplate 105 into the intervertebral disc 100.

FIG. 5 shows the superior view of an endplate 105 punctured by the elastically curved needle 101 carrying the conduit 126.

FIG. 6 shows retrieval of the elastically curved needle 101 into the rigid needle 220. The plunger 109 has been held stationary to deploy the conduit or shunt 126 bridging the vertebral body 159 to the disc 100.

FIG. 7 shows the top view of the endplate shunt or conduit 126 after retrieval of the elastically curved needle 101 into the rigid needle 220.

FIG. 8 depicts an anterior approach for implanting the endplate shunt 126 by retracting the blood vessels 112 and drilling through the vertebral body 159 toward the middle of the endplate 105.

FIG. 9 shows the side view of the drilling of the vertebral body 159 toward the center of the endplate 105. The drill bit 150 contains a drill stop 279 to prevent excessive drilled depth.

FIG. 10 shows a needle 101 carrying a conduit 126 and plunger 109 puncturing through the endplate 105 into the disc 100 to deliver the endplate shunt 126.

FIG. 11 shows the endplate shunt 126 bridged between interior of the vertebral body 159 and the disc 100.

FIG. 12 shows a flexible drill bit 150 with cutting grooves 290, strain-relieving elements 191, shaft 300, base 297, grip 298 and fastener 299.

FIG. 13 shows another flexible drill bit 150 with a thin flexible shaft 300.

FIG. 14 shows another flexible drill bit 150 with a flexible coil as the shaft 300.

FIG. 15 depicts a gear 301 with a drive hole 303 sized and configured to fit the grip 298 of the flexible drill bit 150.

FIG. 16 depicts a flexible drill bit 150 attached to the gear 301 driven by a second gear 302 connected to a crank handle 305.

FIG. 17 shows slits 309 at the distal end of the elastically curved needle 101.

FIG. 18 shows drilling of the calcified endplate 105 by the flexible drill bit 150 positioned, guided or directed by the elastically curved needle 101.

FIG. 19 shows entry of the collapsible slit needle 101 into the drilled hole of the endplate 105.

FIG. 20 depicts a beveled 310 tip of the slit needle 101 to facilitate endplate 105 entry.

FIG. 21 shows multiple slits 309 at the distal end of the elastically curved needle 101.

FIG. 22 shows a conduit 126 and a plunger 109 on a flexible slide 311 with a sharpened tip.

FIG. 23 depicts insertion of the flexible slide 311 carrying the conduit 126 into the pre-drilled hole.

FIG. 24 depicts deployment of endplate conduit 126 by withdrawing the slide 311 and holding the plunger 109 stationary.

FIG. 25 shows a thin drill sleeve 313 over the drill bit 150.

FIG. 26 shows a conduit 126 abutting a plunger 109 exiting from a lumen 312 of a tubular portion of the slide 311.

FIG. 27 depicts advancement of the drill sleeve 313 over the drill 150 after endplate 105 drilling, as shown in FIG. 18.

FIG. 28 shows replacement of the drill 150 with the conduit 126 and slide 311, as shown in FIG. 26, being inserted into the drill sleeve 313.

FIG. 29 shows withdrawal of the drill sleeve 313 into the curved needle 101, exposing the conduit 126 on the slide 311. The conduit 126 is then deployed by withdrawing the slide 311, while holding the plunger 109 stationary.

FIG. 30 shows a drill 150 with cutting elements 315 and a lumen 314 containing the conduit 126 and slide 311.

FIG. 31 shows the slide 311 and plunger 109 extending proximally from the fastener 299 and the grip 298 of the drill 150, shown in FIG. 30.

FIG. 32 shows drilling of the endplate 105 with the cutting elements 315, then the conduit 126 and slide 311 are inserted into the lumen 314 of the drill 150.

FIG. 33 shows horizontally oriented strain-relieving elements 191 of the drill 150.

FIG. 34 shows longitudinally oriented strain-relieving elements 191.

FIG. 35 shows insertion of a trocar 103 to clear debris cored by the cutting elements 315.

FIG. 36 depicts a swellable coating 163 for sealing the gap between the conduit 126 and endplate 105.

FIG. 37 depicts a cone-shaped endplate plug 292 with a lumen 295.

FIG. 38 shows the plug 292 capable of sliding over the needle 101 punctured through the endplate 105.

FIG. 39 shows a plug sleeve 271 pushing the plug 292 into the punctured hole of the calcified endplate 105.

FIG. 40 shows withdrawal of the needle 101 while the sleeve 271 further advancing the plug 292 to seal between the conduit 126 and endplate 105, while the plunger 109 is held stationary to deploy the conduit 126.

FIG. 41 depicts hydration and swelling of the plug 292 sealing the gap between the conduit 126 and the calcified endplate 105.

FIG. 42 shows a cone-shaped endplate plug 292 with a closable slit 293.

FIG. 43 shows the endplate plug 292 being slid over the needle 101 by the plug sleeve 271.

FIG. 44 shows closing of the slit 293 after being slid off the needle 101 to seal the gap between the plug 292 and the calcified endplate 105.

FIG. 45 shows an endplate plug 292 with self-tapping thread 294.

FIG. 46 shows a nut 296 portion for rotating and advancing the plug 292 into the endplate 105.

FIG. 47 shows that a flexible sleeve 271 fits over the nut 296 for advancing the plug 292 over the needle 101.

FIG. 48 shows a cross-section of the plug 292, nut 296, plug lumen 295, slit 293, needle 101 and conduit 126.

FIG. 49 shows the cross-section after withdrawal of the needle 101 and closure of the slit 293 to seal the conduit 126 within the lumen 295 of the plug 292 in the endplate 105.

FIG. 50 shows shape distortion of the nut 296 after slit 293 closure, creating free spinning of the sleeve 271 to prevent excessive plug 292 tightening into the endplate 105.

FIG. 51 shows puncture sites 152, marked by two “X” marks, for implanting an endplate shunt 126 through multiple discs 100.

FIG. 52 depicts the compliant nature of the colon 119. A rod 144 through the rectum 111 cannot reposition the colon 119 to allow insertion of the obturators 141.

FIG. 53 shows a colon positioner 145 equipped with a vacuum line 149 and a suction cup 146 for holding or lifting the inner lining of the colon 119.

FIG. 54 shows vacuum suction of the positioner 145 lifting the colon 119 to provide entries to the obturators 141 within the sheaths 230.

FIG. 55 shows replacements of obturators 141 with a drill 150 and an endoscope 117, drilling superiorly into vertebral bodies S1 to as high as L3.

FIG. 56 shows replacement of the drill with a needle 101 containing a long conduit 126 abutted against a plunger 109.

FIG. 57 shows deployment of the conduit 126 by withdrawing the needle 101 while holding the plunger 109 stationary. The conduit 126 re-establishes exchange of nutrients and waste for multiple discs 100.

### DETAILED DESCRIPTION OF THE EMBODIMENTS

Pedicle 278 puncturing with a trocar can be guided by a fluoroscope, ultrasound or MRI. The trocar can also be coated with radiopaque, echogenic or magnetic coating to intensify the image. A tubular dilator is inserted over the trocar. The trocar is then replaced with a drill, which drills into the pedicle 278 toward the center of the vertebral body 159.

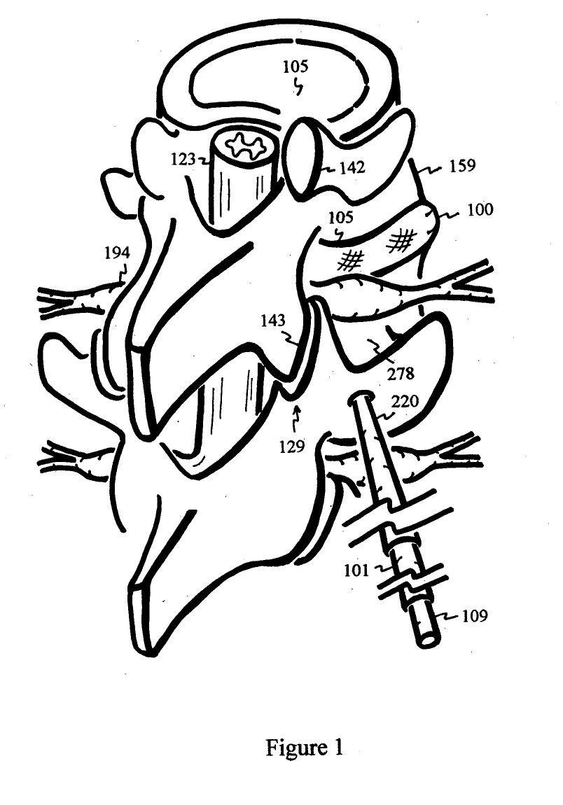

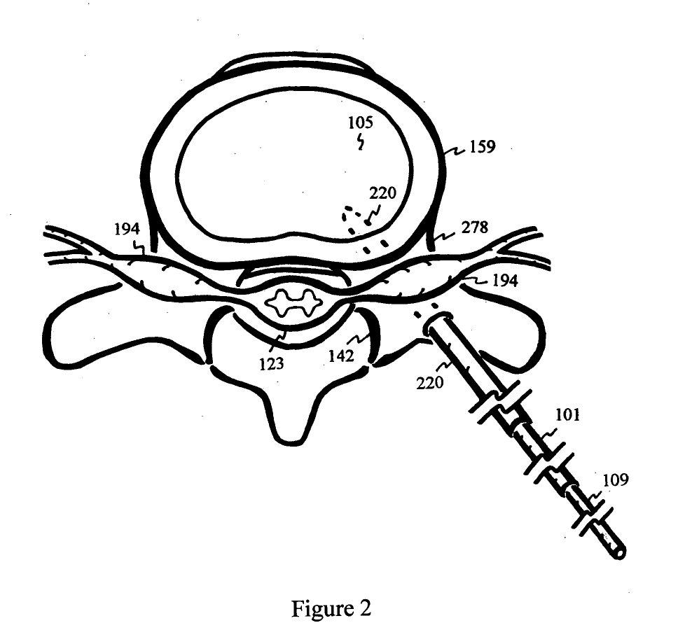

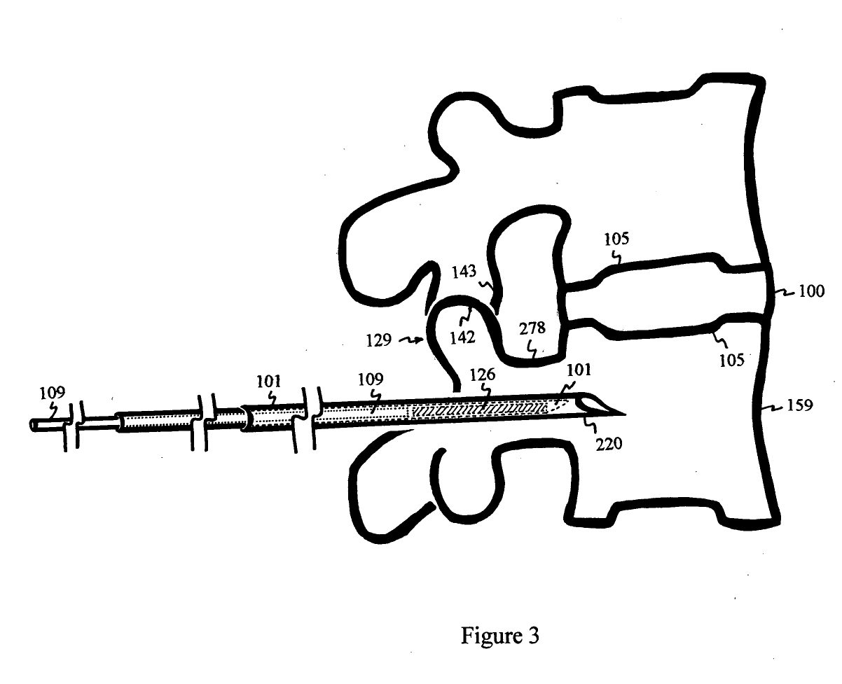

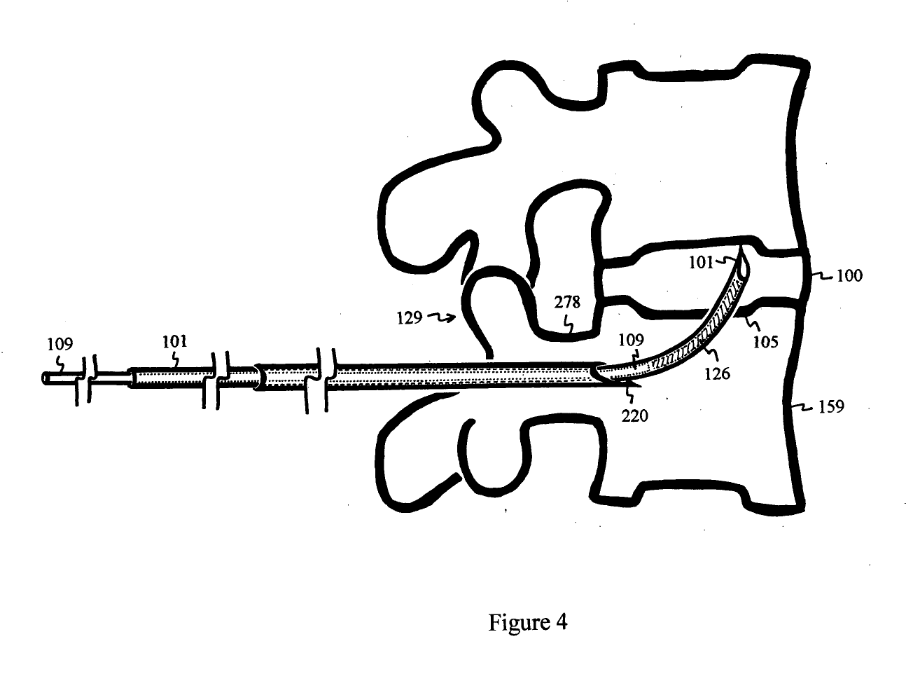

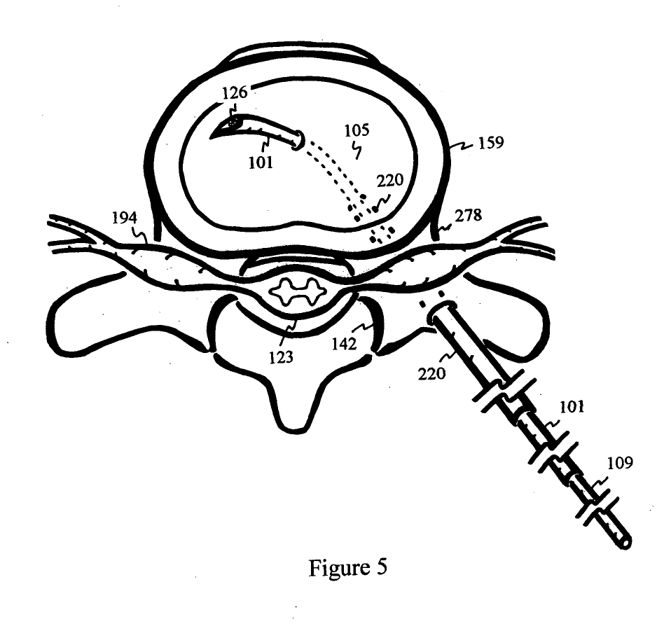

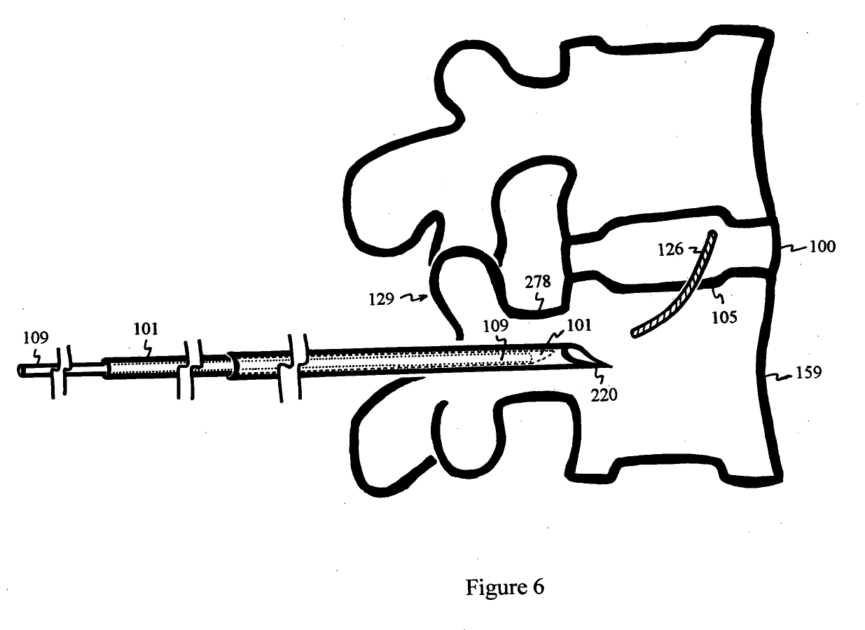

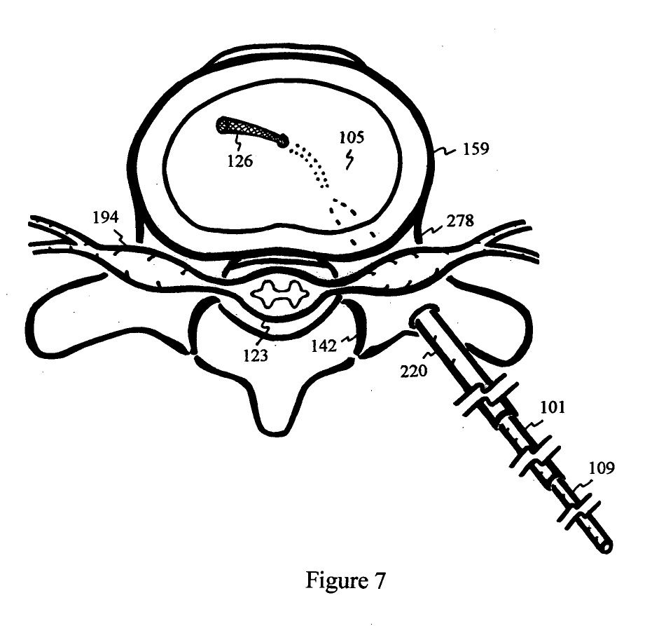

The drill is replaced with a conduit 126 delivery device. The delivery device contains a conduit 126 abutted against a plunger 109 within an elastically curved needle 101. The elastically curved needle 101 is resiliently straightened within a rigid needle 220. FIG. 1 shows insertion of the conduit 126 delivery device through the dilator, not shown, into the pedicle 278. The pedicle 278 puncturing circumvents the iliac blockage and prevents potential injury to the nerve 194, as shown in FIG. 2. FIG. 3 shows a side view of a pedicle 278 puncture into the vertebral body 159 with the rigid needle 220 containing the elastically curved needle 101, conduit 126 and plunger 109. FIG. 4 shows deployment of the elastically curved needle 101 from the rigid needle 220. The elastically curved needle 101 resumes the curvature when deployed from the rigid needle 220 and punctures through the calcified endplate 105 into the intervertebral disc 100. The center of the calcified endplate 105 is usually the thinnest portion; therefore it is a good location for puncturing. FIG. 5 shows the superior view of endplate 105 puncture by the elastically curved needle 101 housing or carrying the conduit 126. The conduit 126 is deployed by retrieving the elastically curved needle 101 into the rigid needle 220 while holding the plunger 109 stationary, as shown in FIG. 6. The conduit 126 is deployed at the endplate 105 bridging between the intervertebral disc 100 and the interior of the vertebral body 159. FIG. 7 shows the superior view of the endplate shunt 126 after retrieval of the elastically curved needle 101 into the rigid needle 220 to deploy the conduit 126. The disc 100 is not shown in FIG. 7.

Discs adjacent to spinal fusion often show rapid degeneration leading to recurrent back pain. Similarly, discs adjacent to a disc replacement may not have degenerated enough to be replaced, but may be vulnerable to becoming a source of recurrent back pain. Disc shunts or conduits 126 can be used in discs 100 adjacent to spinal fusions or disc replacements to slow, stop or reverse disc 100 degeneration.

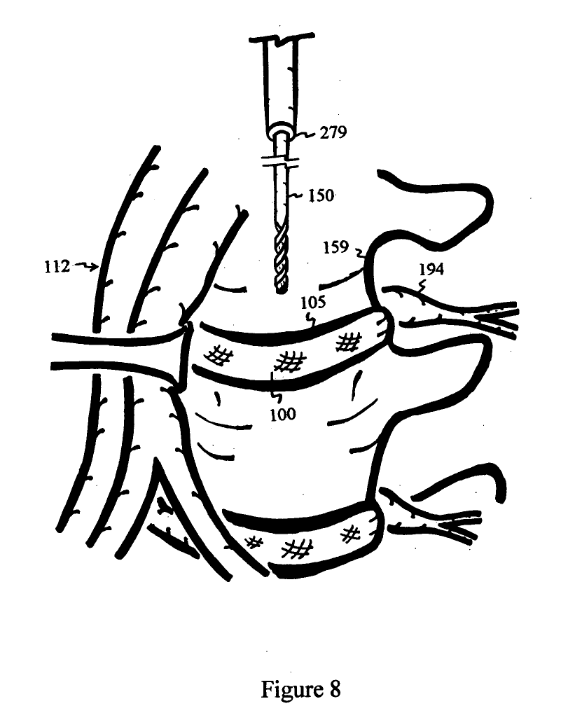

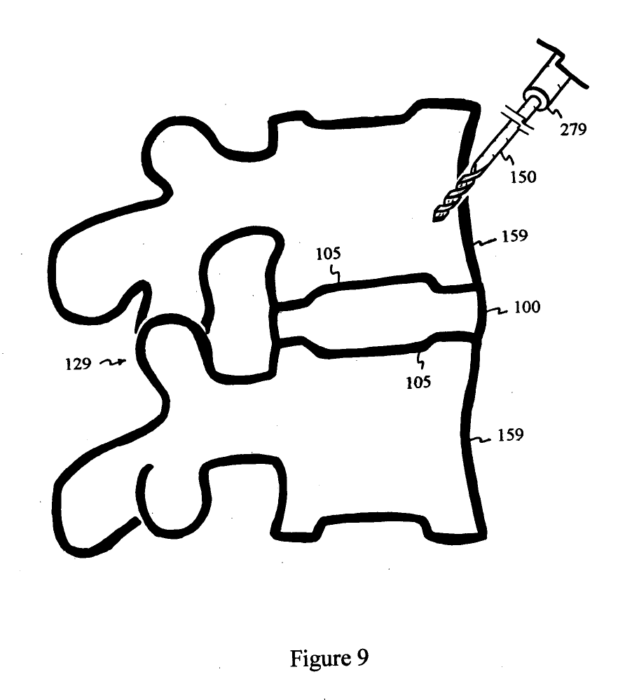

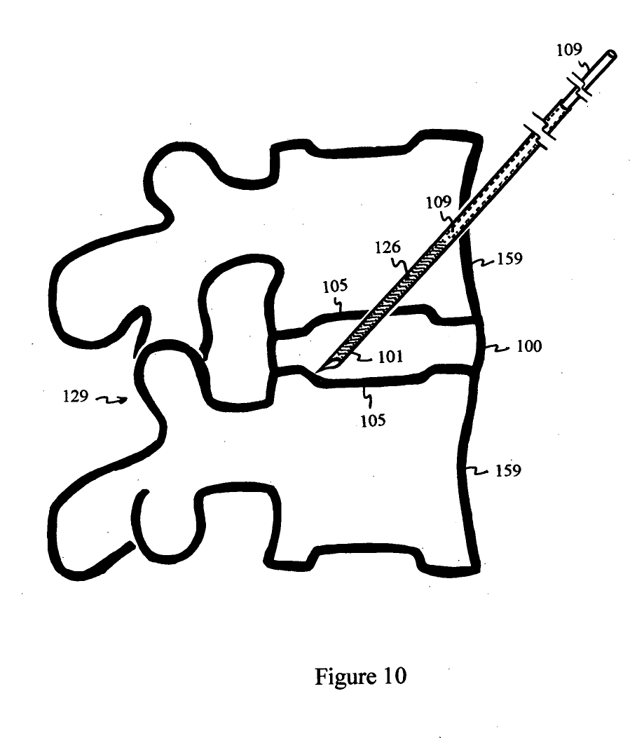

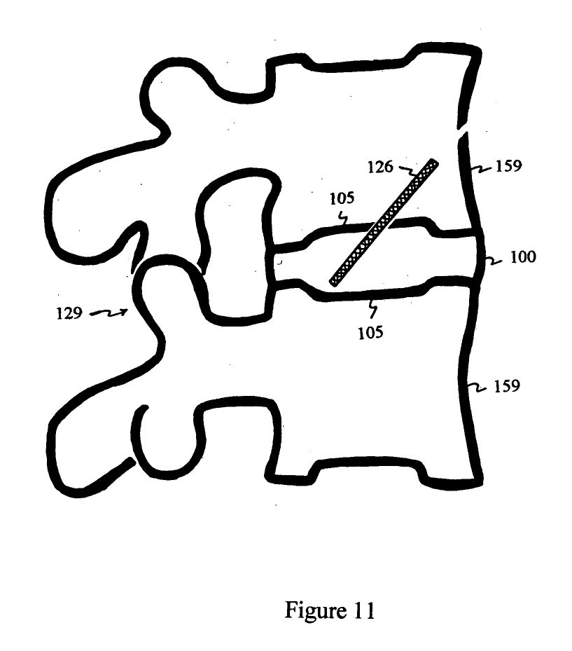

Many spinal fusion and disc replacement procedures use anterior approaches. Since the patient is already open, blood vessels 112 can be retracted to expose the vertebral body 159, as shown in FIG. 8. A drill 150 is used to penetrate through the vertebral body 159 toward the center of the adjacent endplate 105. The drill bit 150 contains a drill stop 279 to prevent drilling too deeply. FIG. 9 shows a side view of a vertebral body 159 being drilled toward the center of the endplate 105. The drill bit 150 is replaced by a straight needle 101 containing a conduit 126 abutted by a plunger 109, as shown in FIG. 10. The conduit 126 is deployed, as shown in FIG. 11, by withdrawing the needle 101 while holding the plunger 109 stationary. The conduit 126 becomes an endplate shunt 126 for re-establishing the exchange of nutrients and waste between the interior of the vertebral body 159 and the disc 100.

PCT/US04/14368 (WO 2004/101015) by J. Yeung and T. Yeung on May 7, 2004, also proposed annular shunts 126 across the disc 100 to draw nutrients from the outer annulus into the inner annulus to feed the deprived cells. Annular shunts 126 can also be used to slow, stop or reverse degeneration of discs 100 adjacent to spinal fusion, disc replacement or vertebroplasty to minimize or prevent recurrent back pain.

Pedicle 278 entry is currently being used to infuse bone cement or inflatable devices with a straight needle to repair vertebral fracture. The straight needle is as large as 11-gauge, about 3 mm diameter. The repair with bone cement is called vertebroplasty, which can be an out-patient procedure. Since the passage into the pedicle 278 can be as large as 3 mm in diameter, a stacking of a rigid needle 220, an elastically curved needle 101, a drill bit 150, an endplate plug 292, a plug sleeve 271 and conduit 126 can enter through the pedicle 278. The elastically curved needle 101 is used to carve through the spongy cancellous bone within the vertebral body 159, toward the calcified endplate 105. The elastically curved needle 101 can curve superiorly or inferiorly to implant conduits 126 in the endplates 105 above and below the pedicle 278.

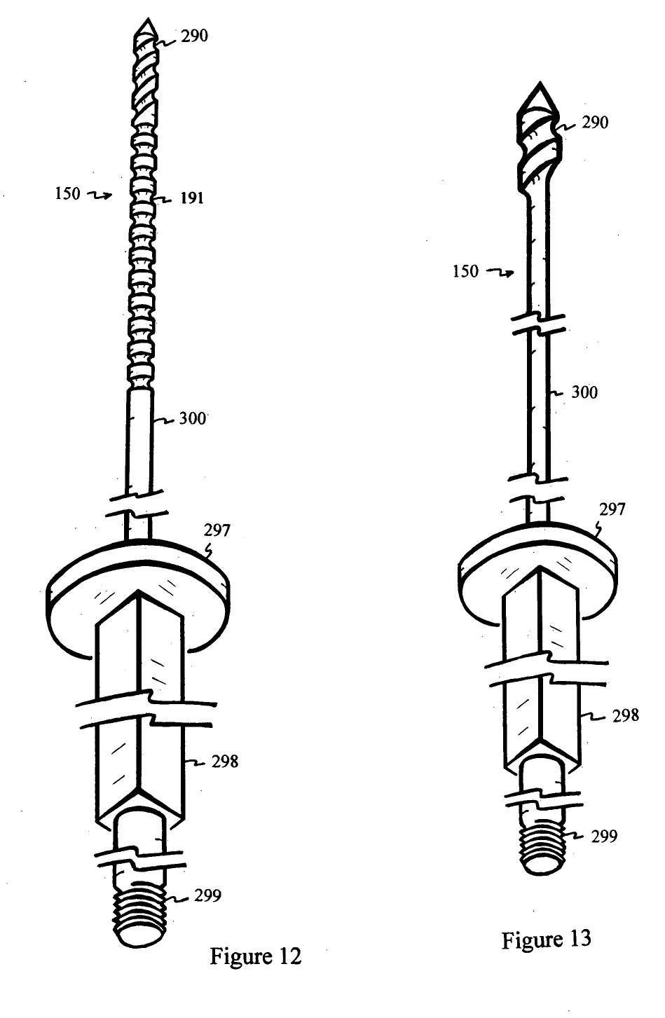

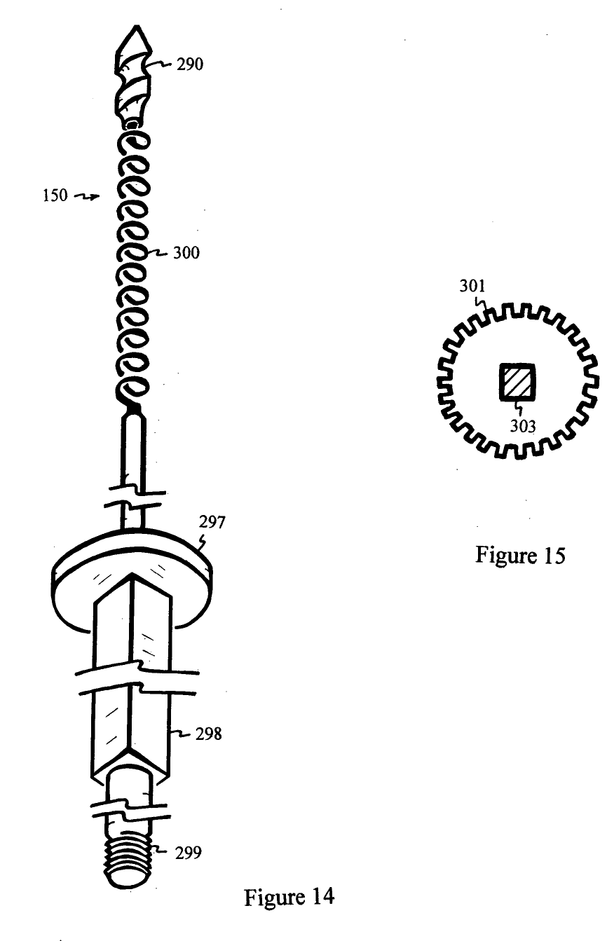

Calcified endplates 105 can be hard to puncture with a needle 101. Flexible drill bits 150 are proposed for drilling through the endplate 105 prior to conduit 126 insertion. Since the thickness of cartilaginous endplate 105 is only between 0.5 and 2.5 mm, drilling through the endplate 105 is not difficult. FIG. 12 shows a flexible drill bit 150 with cutting grooves 290, strain-relieving elements 191, shaft 300, base 297, grip 298 and fastener 299. The strain-relieving elements 191 provide stress and strain relief when operating under curved or flexed conditions. The shaft 300 can be made thin to improve flexibility, as shown in FIG. 13. The shaft 300 can also be a coil, as shown in FIG. 14, to improve drilling capability in a curved condition. The base 297, grip 298 and fastener 299 are used to mount the drill bit 150 to a drilling mechanism. The flexible drill bit 150 may also contain a widened section as a drill stop to prevent excessive depth of drilling. Drill depth can also be limited by the length of the drill bit 150.

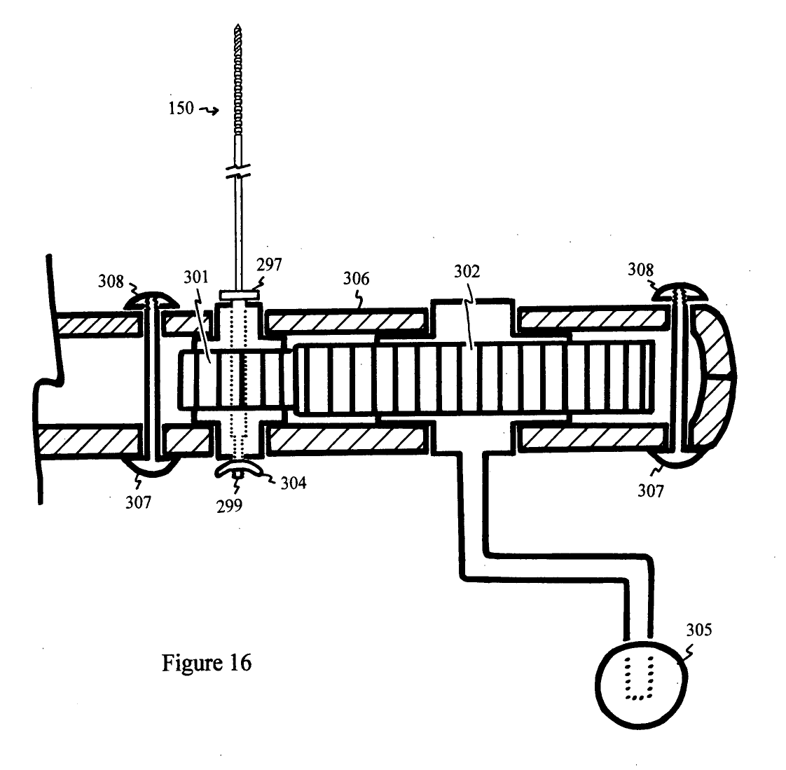

FIG. 15 depicts a gear 301 with a drive hole 303 sized and configured to fit the grip 298 of the flexible drill bit 150. The base 297 of the drill bit 150 is used to rest or press against the gear 301. The grip 298 is inserted into the drive hole 303 of gear 301 and fastened by a wing nut 304 onto the fastener 299 of the drill bit 150, as shown in FIG. 16. The gear 301 can be driven by a second gear 302 connecting to a crank handle 305. Both gear 301 and the second gear 302 are engaged within a drill housing 306 held together by bolts 307 and nuts 308, as shown in FIG. 16.

The flexible drill bits 150 can be made with elastic alloy, such as nickel-titanium or spring tempered stainless steel. Since endplate 105 drilling is light duty, the drill bit 150 can be made with a polymer, such as poly-ether-ether-ketone, acetal resin, polysulfone, polycarbonate, polypropylene, polyethylene, polyamide or other suitable material.

The drill bits 150 can be made by molding, CNC machining, water jet machining, grinding, centerless grinding or other technique. If the drill bit material is metallic, electric discharging machining can be used. The drill bit 150 can also be assembled from modular parts. The parts can be made with different materials to meet various physical requirements.

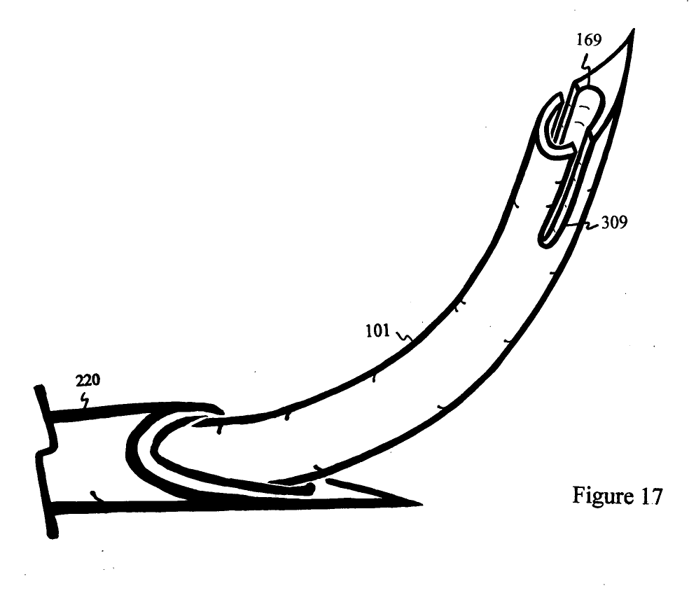

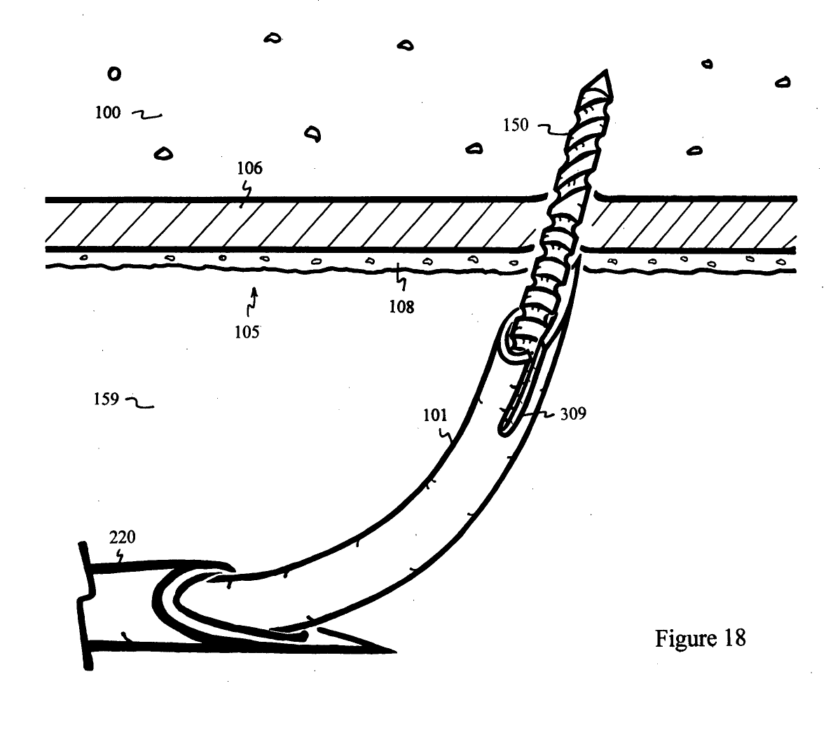

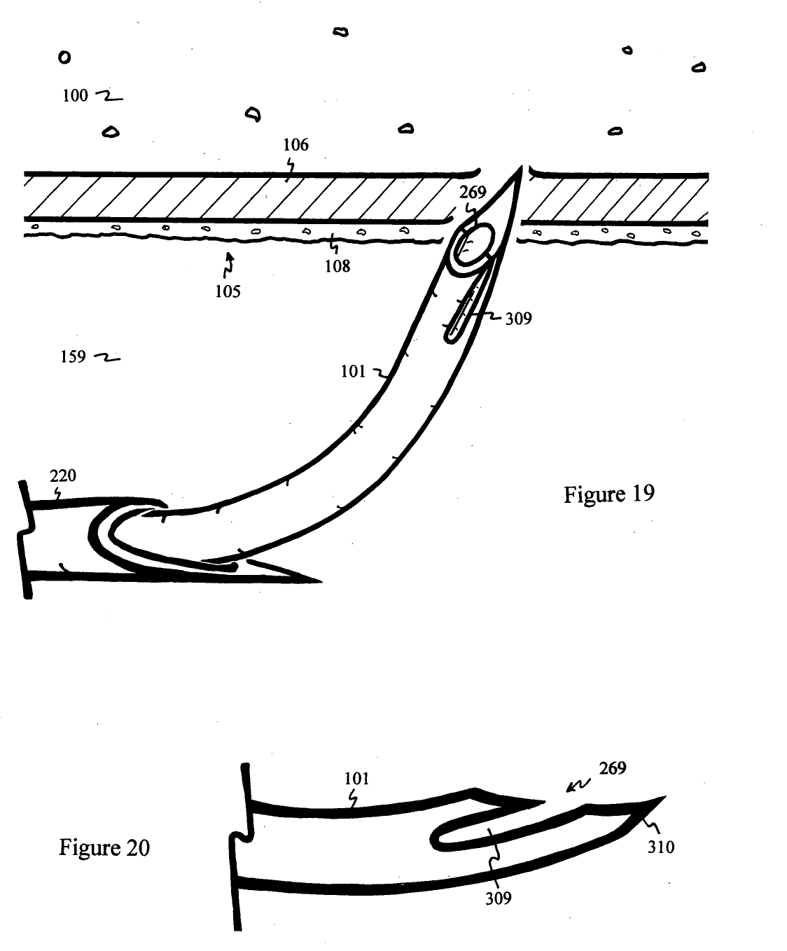

Slits 309 are open at the distal end of the elastically curved needle 101, as shown in FIG. 17. The curved needle 101 is deployed and positioned at the calcified endplate 105. The flexible drill bit 150 is inserted into and guided by the curved needle 101 to drill through the calcified endplate 105, as shown in FIG. 18. After drilling, the curved needle 101 advances into the drilled hole as the flexible drill bit 150 is withdrawn from the drilled hole. The slits 309 allow the diameter of the distal end of the needle 101 to partially collapse or narrow. The needle 101 is positioned at the drilled hole and partially penetrates into the endplate 105, as shown in FIG. 19. A beveled 310 tip tapering or thinning at the outer surface, as shown in FIG. 20, facilitates needle 101 insertion into the drilled hole of the calcified endplate 105. After fixation of the needle 101 at the endplate 105, the drill bit 150 is withdrawn from the needle 101. FIG. 21 shows multiple slits 309 and a beveled 310 tip to further facilitate insertion into and fixation at the hole created at the calcified endplate 105.

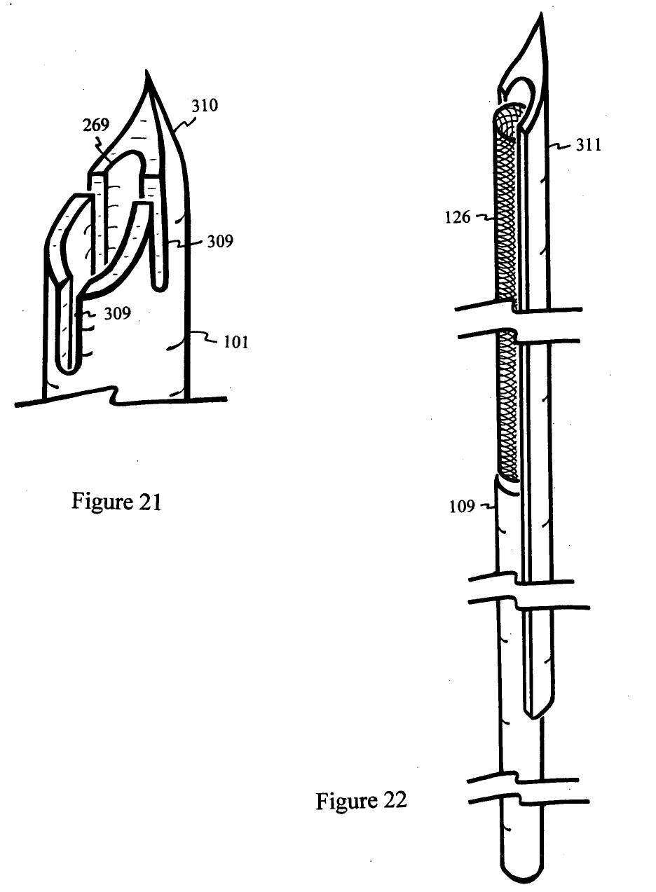

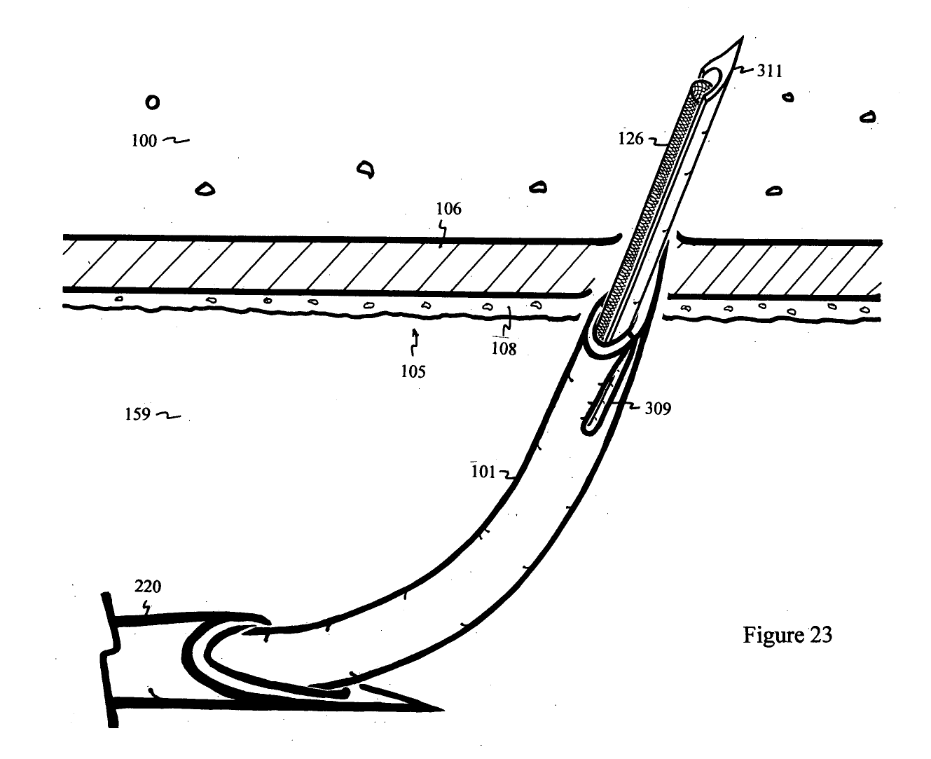

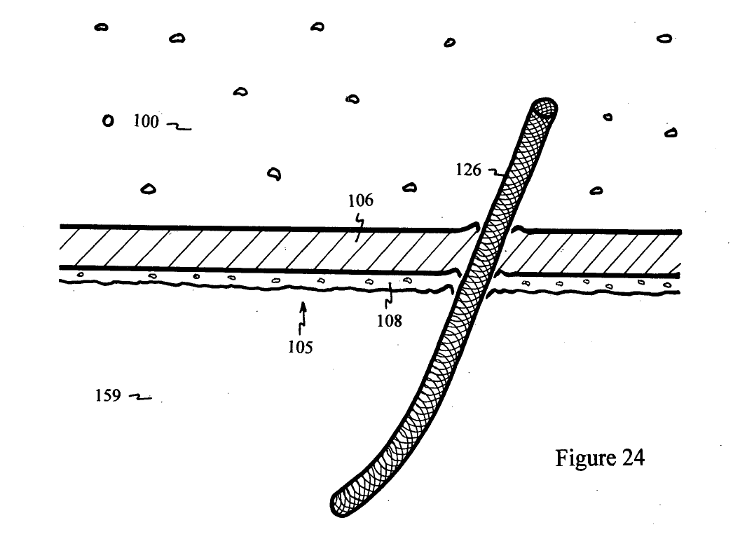

FIG. 22 shows a conduit 126 abutting a flexible plunger 109 on a flexible slide 311 with a sharp distal end. The assembly of the conduit 126, plunger 109 and flexible slide 311 is inserted into the curved needle 101 leading into the drilled hole of the calcified endplate 105 into the intervertebral disc 100, as shown in FIG. 23. The conduit 126 is 10 deployed at the calcified endplate 105 by withdrawing the slide 311 while holding the plunger 109 stationary, as shown in FIG. 24. The deployed conduit 126 bridges between the interior of the vertebral body 159 and the disc 100 to draw nutrients and oxygen from the vertebral body 159 and to feed the deprived cells in the disc 100. In addition, during compressive loading, lactic acid produced within the disc 100 is expelled through the conduit 126 into bodily circulation to normalize the pH within the degenerative disc 100.

The slide 311 provides dual functions: (1) punctures the drill hole into the intervertebral disc 100, and (2) smoothly deploys the conduit 126. Braided material of the conduit 126 can bunch up and jam within a tubular structure, such as the needle 101. The slide 311 provides a stationary semi-cylindrical surface for the conduit 126, reducing the friction between the braided conduit 126 and the needle 101. Hence, the possibility of bunching and jamming of the conduit 126 within the needle 101 is minimized. In addition, jamming of the conduit 126 within the needle 101 can be freed by rotating the slide 311. The slide 311 can be made from a thin metal or alloy, such as nickel-titanium, stainless steel or spring tempered stainless steel. The slide 311 can also be made with polymer. The cross-section of the slide 311 can be a fraction of a circle, elliptical or another shape.

An ultra thin and flexible tube can also be used to contain the conduit 126, slide 311 and plunger 109. The assembly of the ultra thin tube, conduit 126, slide 311 and plunger 109 inserts into the needle 101, through the drilled hole of the calcified endplate 105 into the disc 100. The conduit 126 is deployed by withdrawing the ultra thin tube, followed by the slide 311 while holding the plunger 109 stationary.

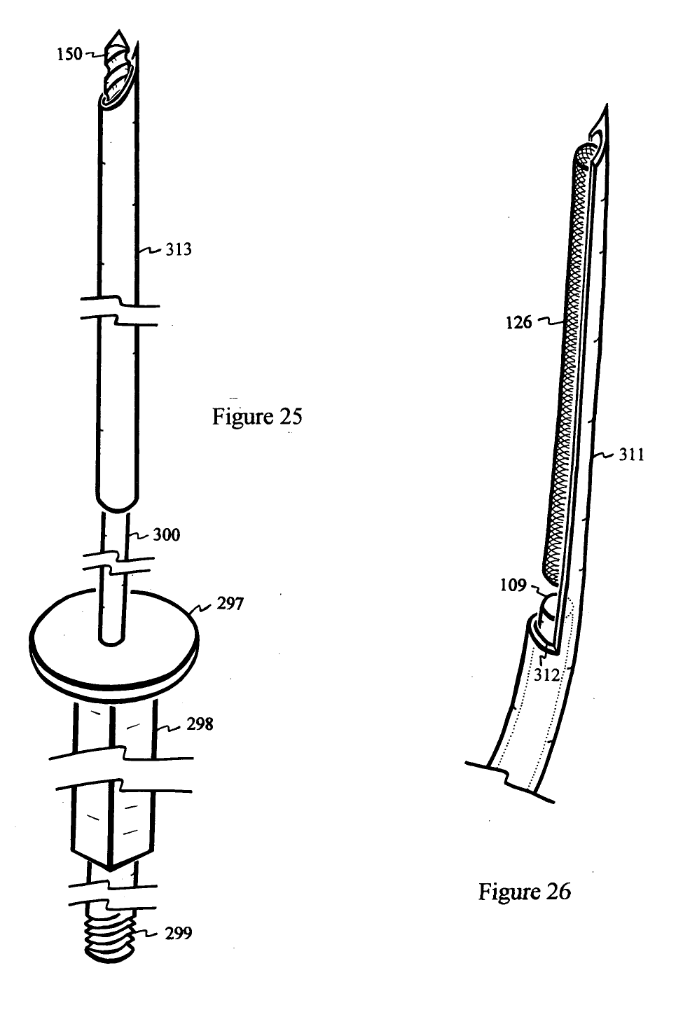

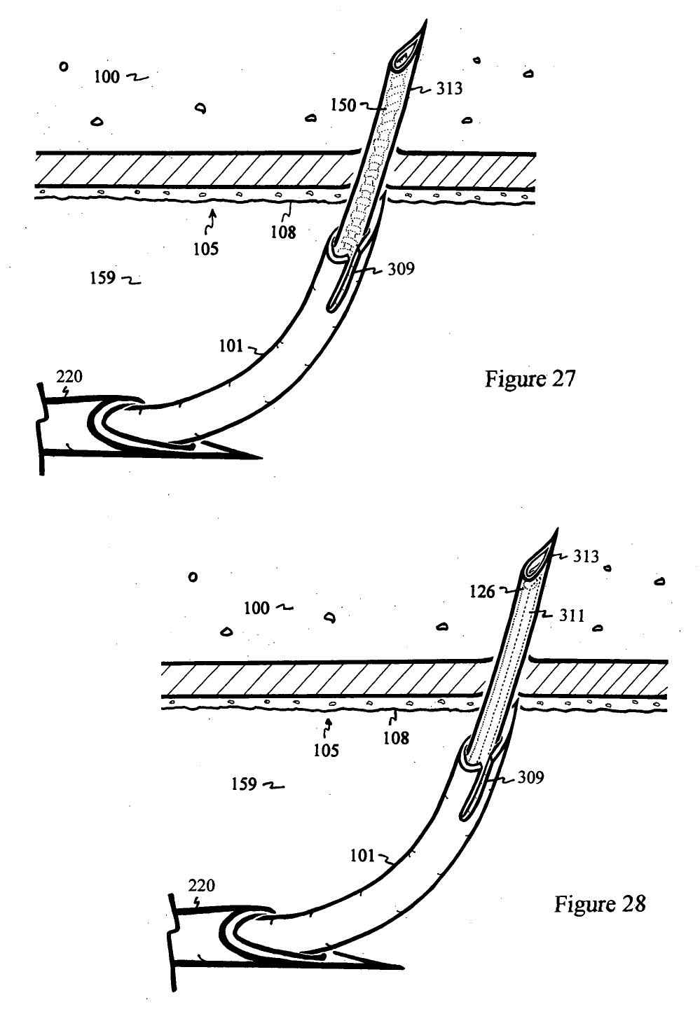

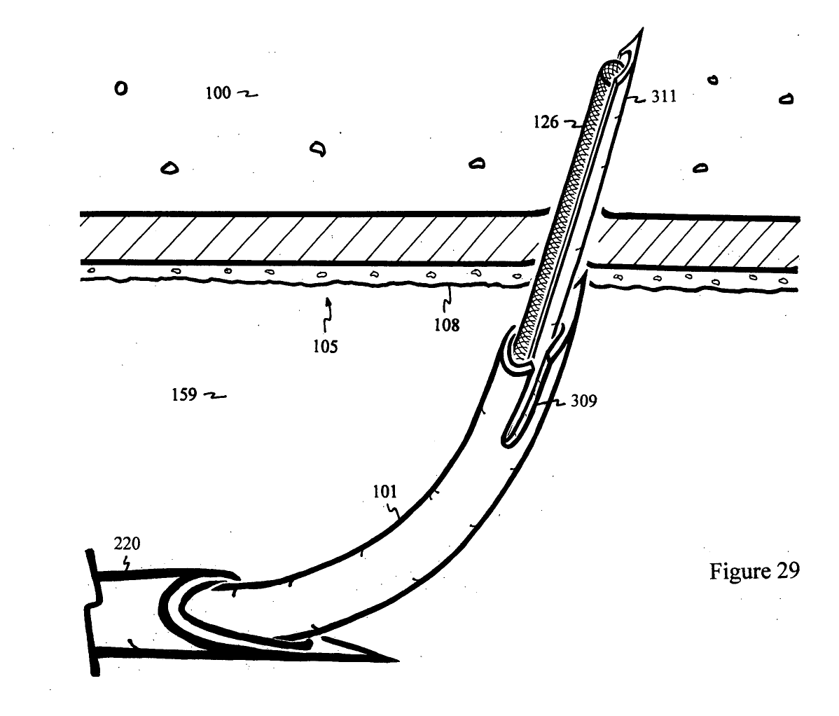

A thin, flexible drill sleeve 313 can be used to maintain the drilled position at the endplate 105. FIG. 25 shows the flexible drill sleeve 313 with a sharp distal end, sliding over the drill bit 150. FIG. 26 shows a modified slide 311 with a trough at the distal end, a plunger 109 within the lumen 312 of the tubular, proximal end of the slide 311. After endplate 105 drilling, the flexible drill sleeve 313 slides over the drill bit 150 through the drilled hole into the disc 100, as shown in FIG. 27. The drill bit 150 is replaced by the assembly of the conduit 126, slide 311 and plunger 109, as shown in FIG. 28. The drill sleeve 313 is retrieved, exposing the conduit 126 and the slide 311, as shown in FIG. 29. The conduit 126 is then deployed at the calcified endplate 105 by withdrawing the slide 311 while holding the plunger 109 stationary.

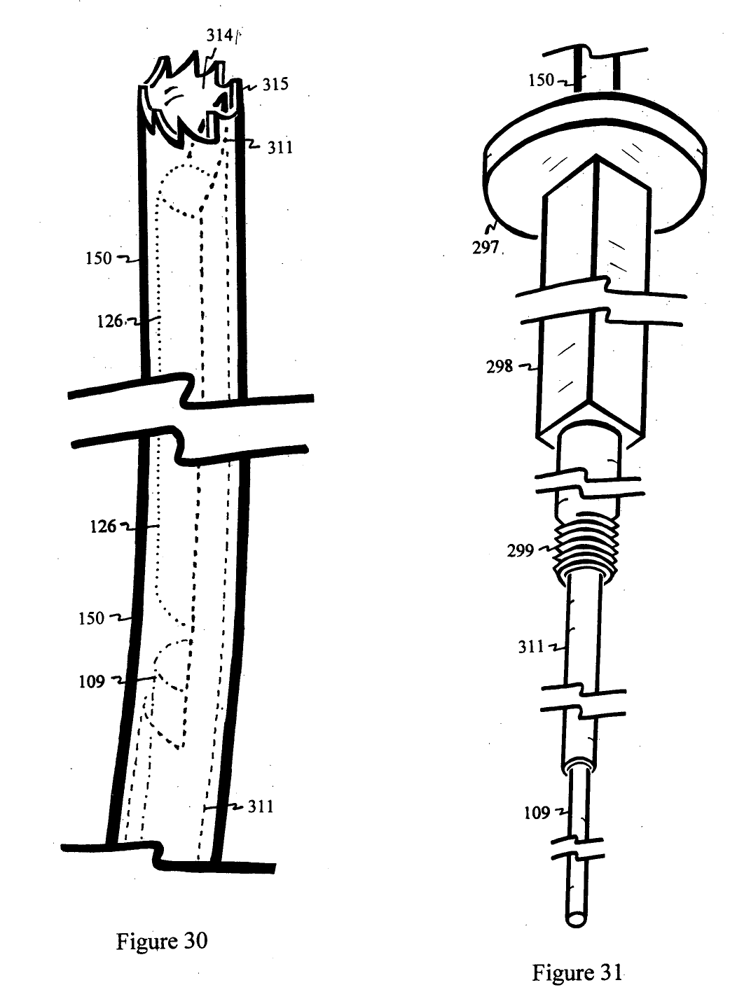

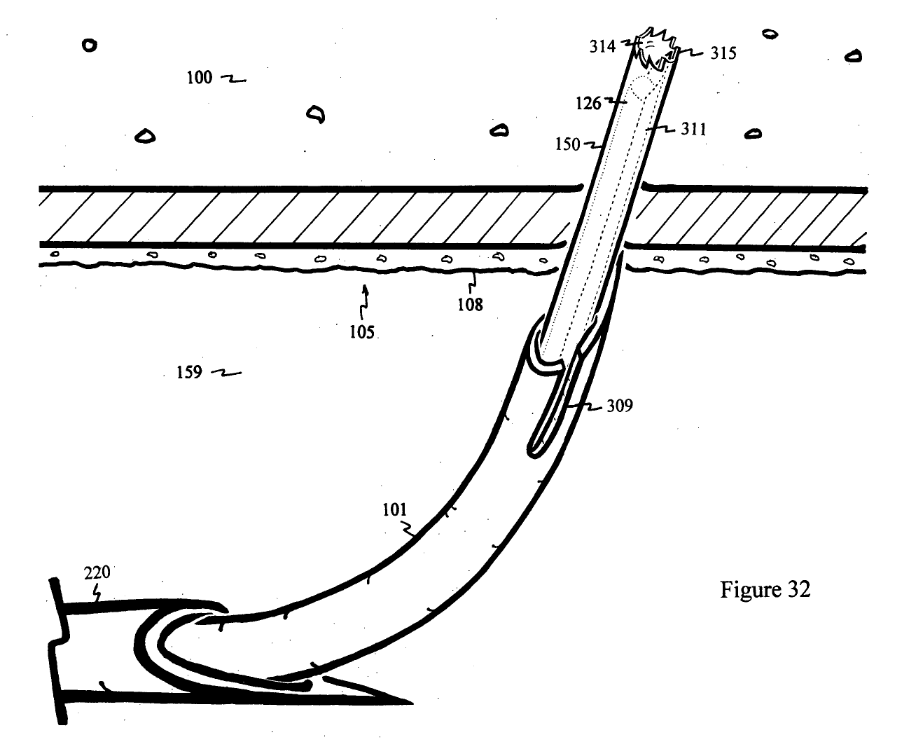

The flexible drill bit 150 can also contain cutting elements 315 and a lumen 314 for passing the conduit 126, slide 311 and plunger 109, as shown in FIG. 30. FIG. 31 shows the proximal ends of the slide 311 and plunger 109 extending from the proximal end of the drill 150 assembly. The flexible drill 150 is guided by the elastically curved needle 101 to drill and cut through the calcified endplate 105 into the intervertebral disc 100. The assembly of conduit 126, slide 311 and plunger 109 inserts into the lumen 314 of the drill bit 150, as shown in FIG. 32. The drill 150 is withdrawn, followed by the slide 311 while holding the plunger 109 stationary to deploy the conduit 126 at the calcified endplate 105.

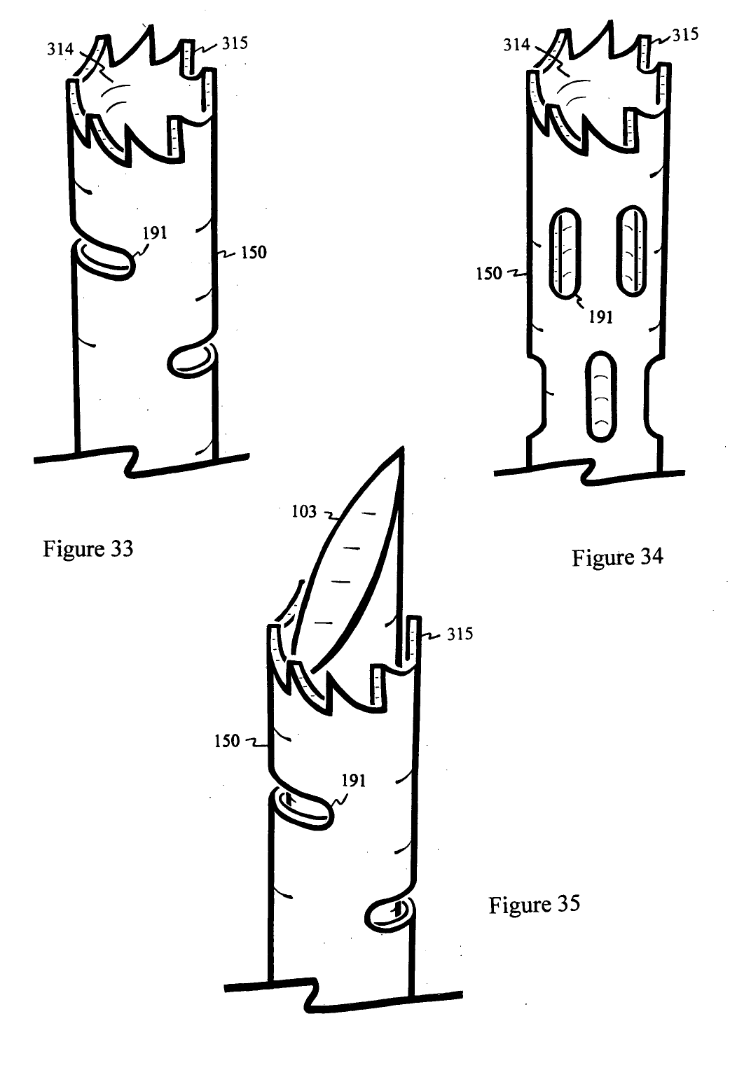

Indentations of the drill shaft 300 in FIG. 12 form the strain-relieving elements 191 for operating under curved or flexed conditions. The strain-relieving elements 191 of the drill 150 can also be a variety of openings. FIG. 33 shows horizontal openings as strain-relieving elements 191. FIG. 34 shows longitudinal openings as strain-relieving elements 191. The strain-relieving elements 191 can also be oriented in other directions. FIG. 35 shows a trocar 103 clearing the debris cored out by the cutting elements 315 of the drill 150. The trocar 103 or the assembly of conduit 126 and slide 311 can advance through the lumen 314 of the drill 150 by rotation to avoid snagging of the strain-relieving element 191.

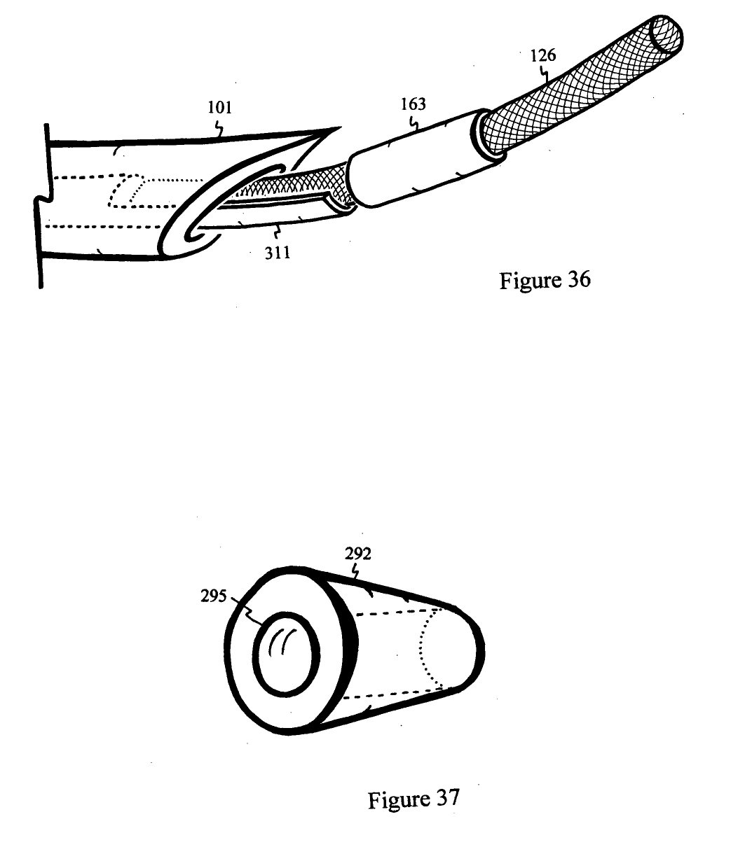

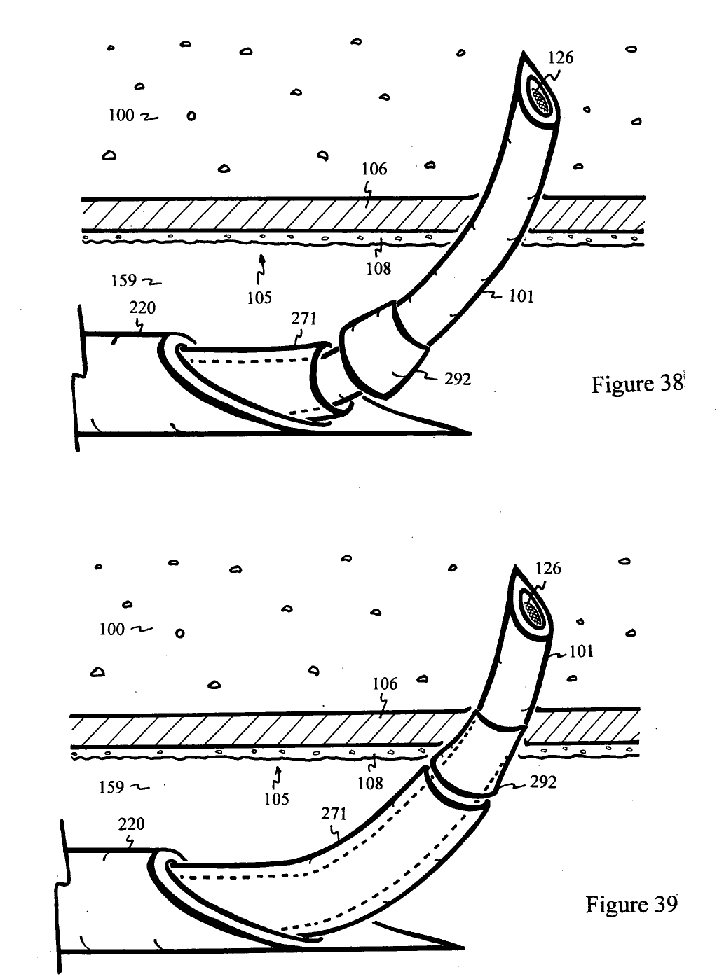

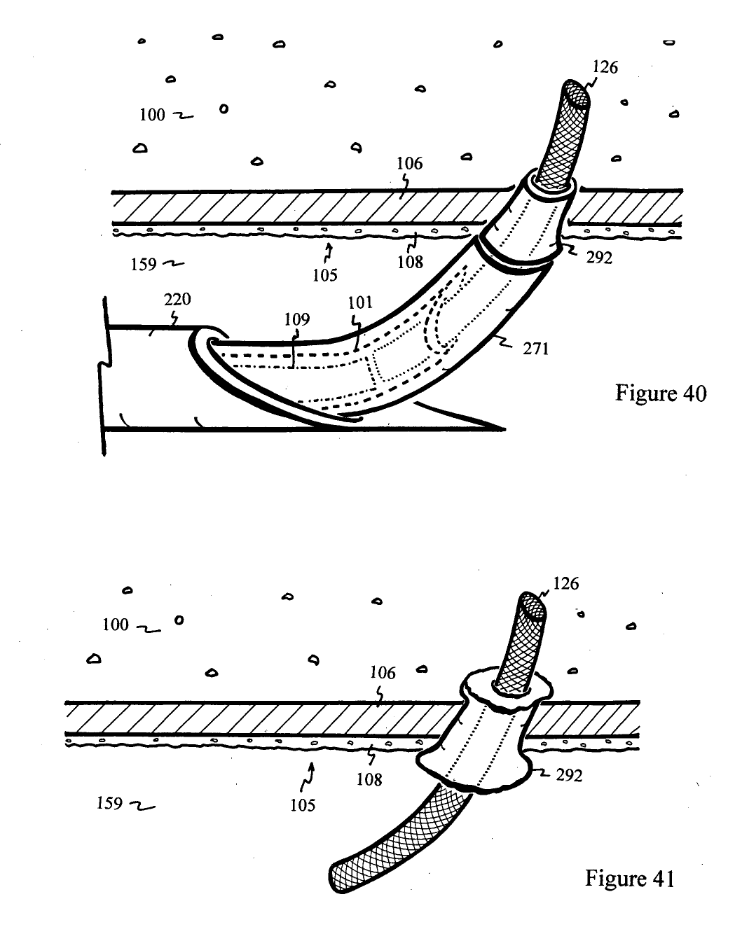

Sealing the gap between the conduit 126 and the endplate 105 prevents immune responses to the nucleus content of the disc 100. In addition, the sealing also preserves the hydrostatic pressure of the disc 100, funneling the flow of nutrients and oxygen through the semi-permeable conduit 126 deep into the avascular disc 100. FIG. 36 shows a swellable coating 163 during hydration to seal the gap between the conduit 126 and the calcified endplate 105. FIG. 37 depicts an elastic or compressible cone-shaped endplate plug 292 with a lumen 295. The wall of the plug 292 is tapered. The lumen 295 of the endplate plug 292 is sized to fit over the elastically curved needle 101, as shown in FIG. 38. After the endplate 105 is punctured, a plug sleeve 271 pushes the plug 292 into the punctured hole of the calcified endplate 105, as shown in FIG. 39. The needle is withdrawn while the sleeve 271 further advances the plug 292 to seal the gap between the conduit 126 and endplate 105, as shown in FIG. 40. The conduit 126 is deployed by retrieving the elastically curved needle 101 while holding the plunger 109 stationary. FIG. 41 depicts hydration and swelling of the plug 292 sealing the gap between the conduit 126 and the calcified endplate 105 to maintain isolation of the nucleus pulposus and preserve the hydrostatic pressure within the disc 100.

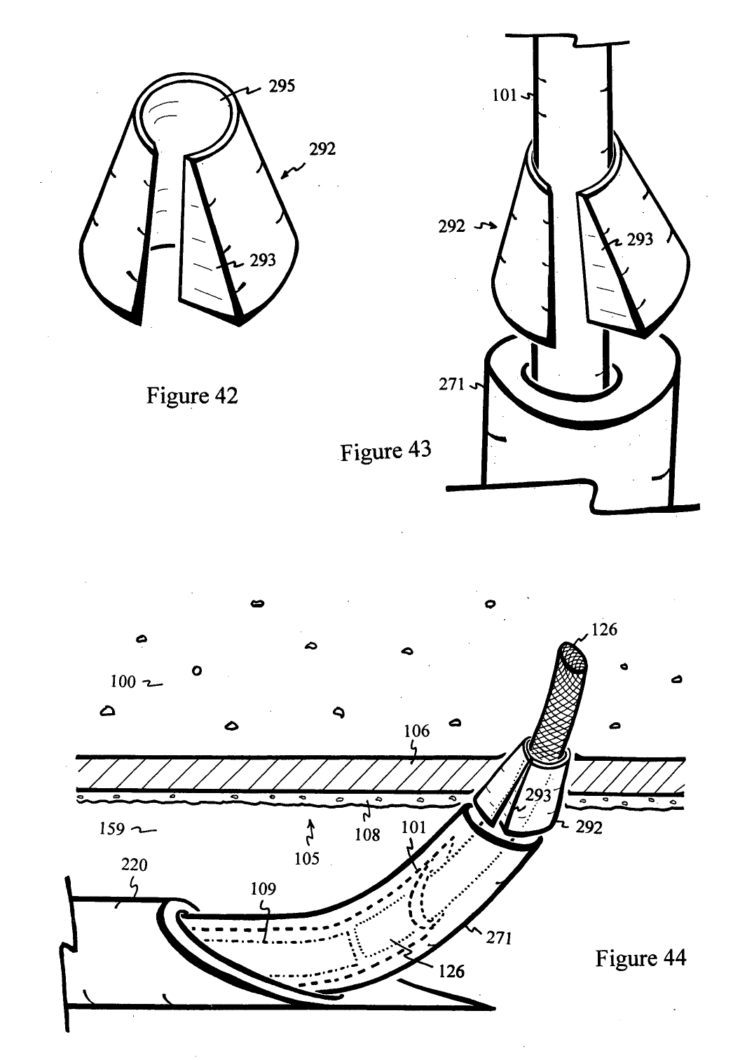

FIG. 42 shows another cone-shaped endplate plug 292 with a closable slit 293. The plug 292 with the slit 293 can also be elastic, compressible and able to slide over the needle 101 by the plug sleeve 271, as shown in FIG. 43. As the plug 292 slides off from the needle 101 into the hole of the endplate 105, the slit 293 closes to provide a tight seal between the conduit 126 and the plug 292, as shown in FIG. 44. The cone-shape and elasticity of the plug 292 provide a tight seal between the plug 292 and the calcified endplate 105.

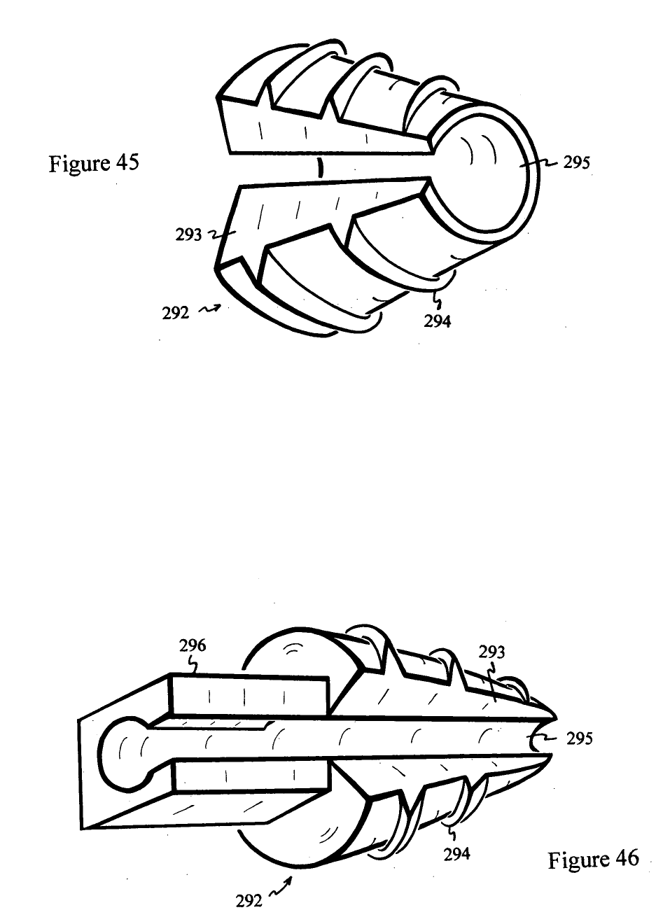

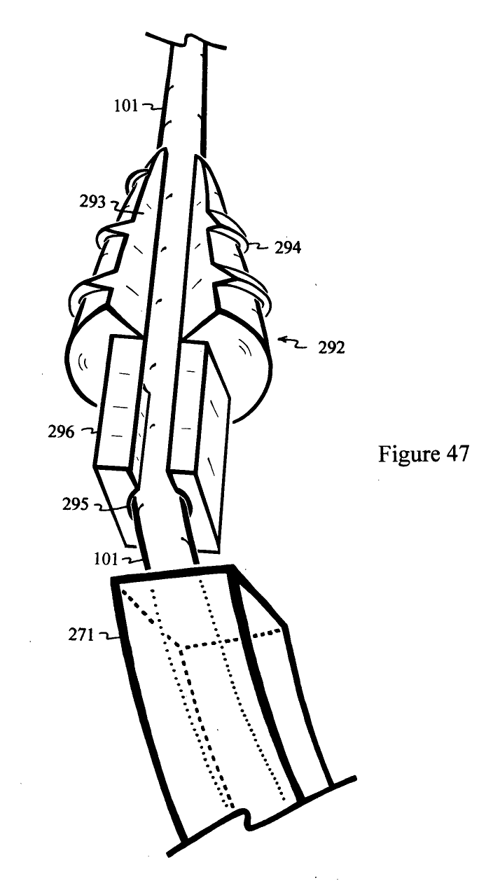

The plug 292 can also contain ridges or self-tapping threads 294 and the slit 293, as shown in FIG. 45. For plug 292 tightening, a nut 296 is formed at the proximal end of the plug 292, as shown in FIG. 46. The slit 293 and lumen 295 extend the entire length of the endplate plug 292, including the nut 296 portion. A plug sleeve 271 is sized and configured to fit over the nut 296 of the plug 292, as shown in FIG. 47, to advance the plug 292 over the needle 101 by rotation into the calcified endplate 105.

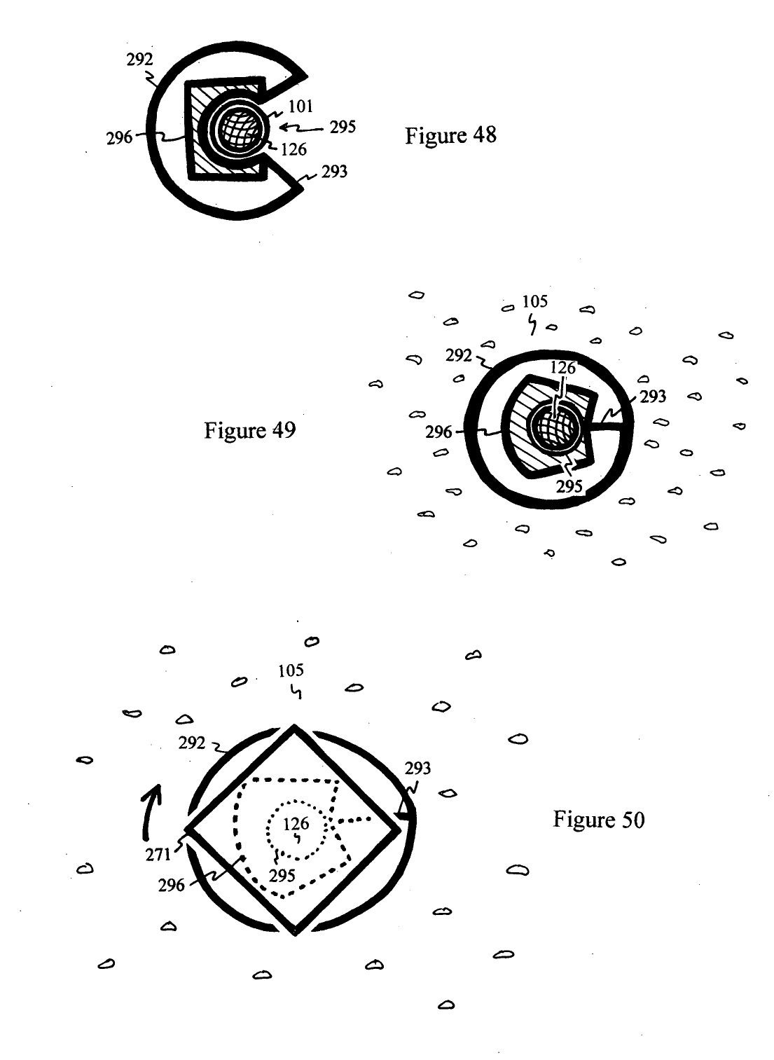

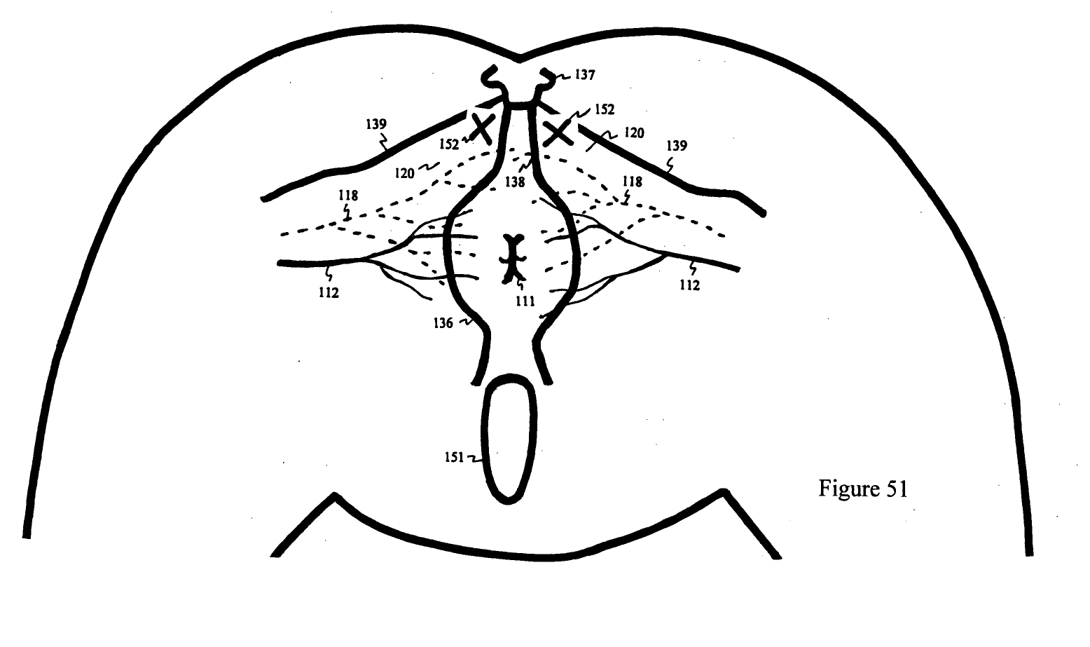

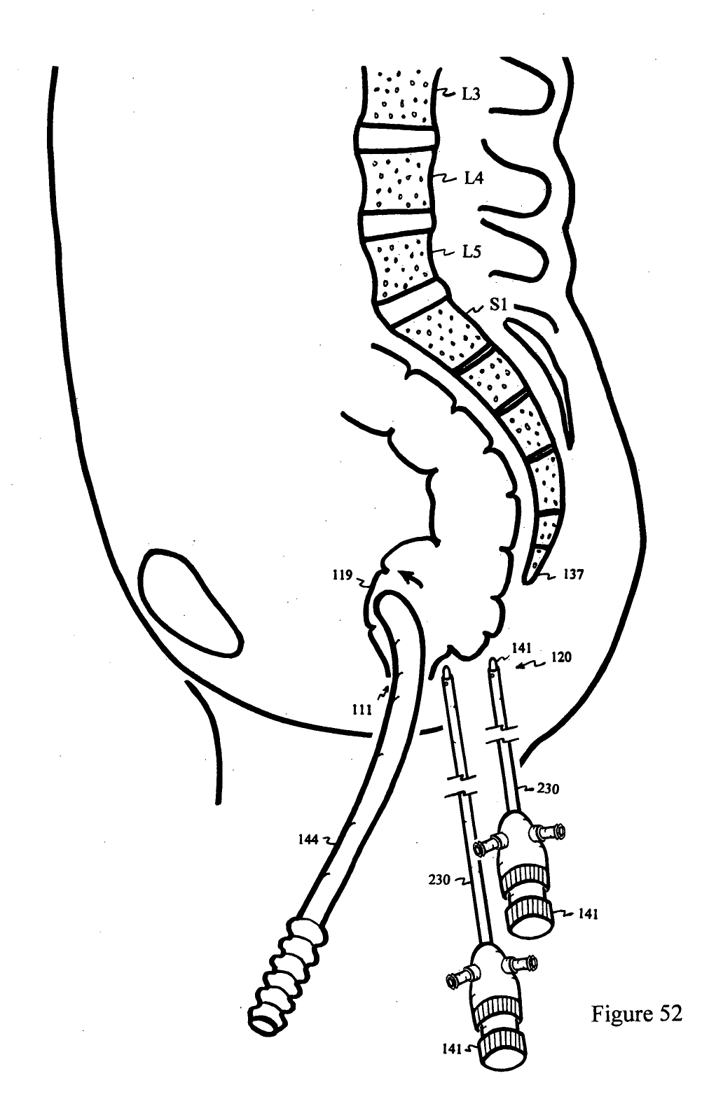

The cross-section of the plug 292, nut 296, plug lumen 295, needle 101 and conduit 126 is depicted in FIG. 48. After the plug 292 is advanced into the endplate 105, the needle 101 is withdrawn and the slit 293 is closed, the lumen 295 of the plug 292 seals around the conduit 126, as depicted in FIG. 49. Upon closure of the slit 293, the cross-section of the nut 296 collapses or shrinks The cross-sectional shape of the nut 296 also becomes distorted or deformed, so the tight fit within the plug sleeve 271 is lost, as shown in FIG. 50. Hence continual rotation of the sleeve 271 will not excessively tighten or advance the plug 292 too deeply into the calcified endplate 105. The cross-section of the nut 296 can be a triangle, square, pentagon, hexagon or other shape along with a matching shape for the sleeve 271 to prevent excessive endplate 105 tightening. The endplate plug 292 can be made with non-degradable or degradable material similar to the one used for the conduit 126.

Back pain may be caused by degeneration of multiple discs 100, which may also explain the common recurrence of back pain shortly after spinal surgery. Many patients experience no pain relief at all after their surgeries. The sacral approach is proposed to implant a conduit 126 through multiple discs 100 using a minimally invasive technique. Punctures 152 can be made through the inferior fascia of the pelvic diaphragm 120, anterior to the coccyx 137 and gluteus maximus muscle 139. Two punctures 152 can be made at both sides of the anococcygeal body 138, as shown in FIG. 51. The nerves 118 and blood vessels 112 are more abundant near the rectum 111, anterior to the punctures 152.

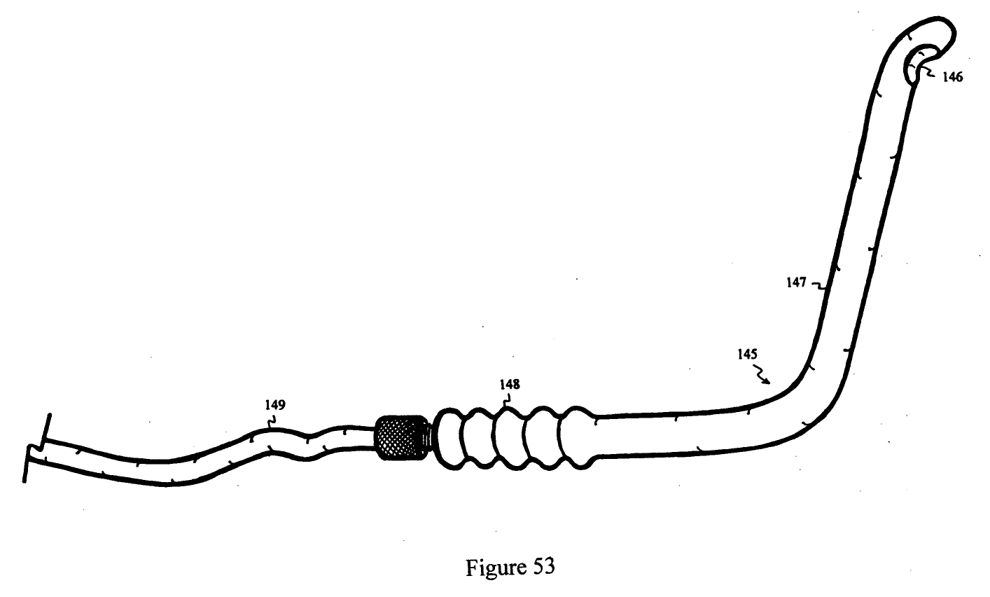

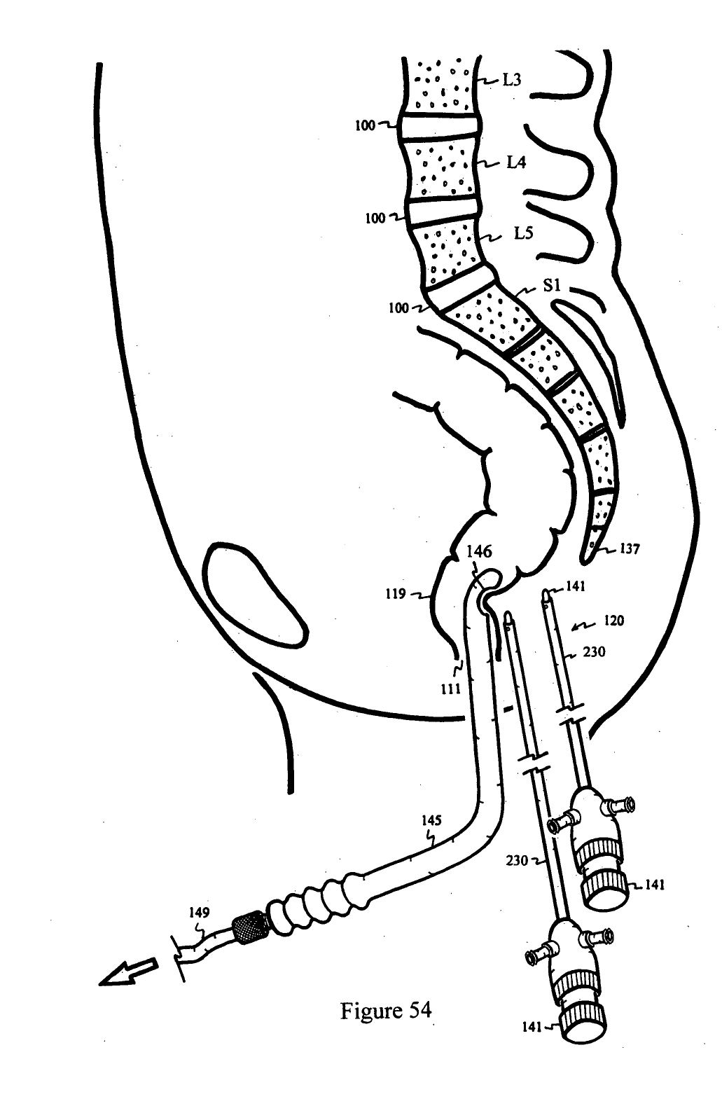

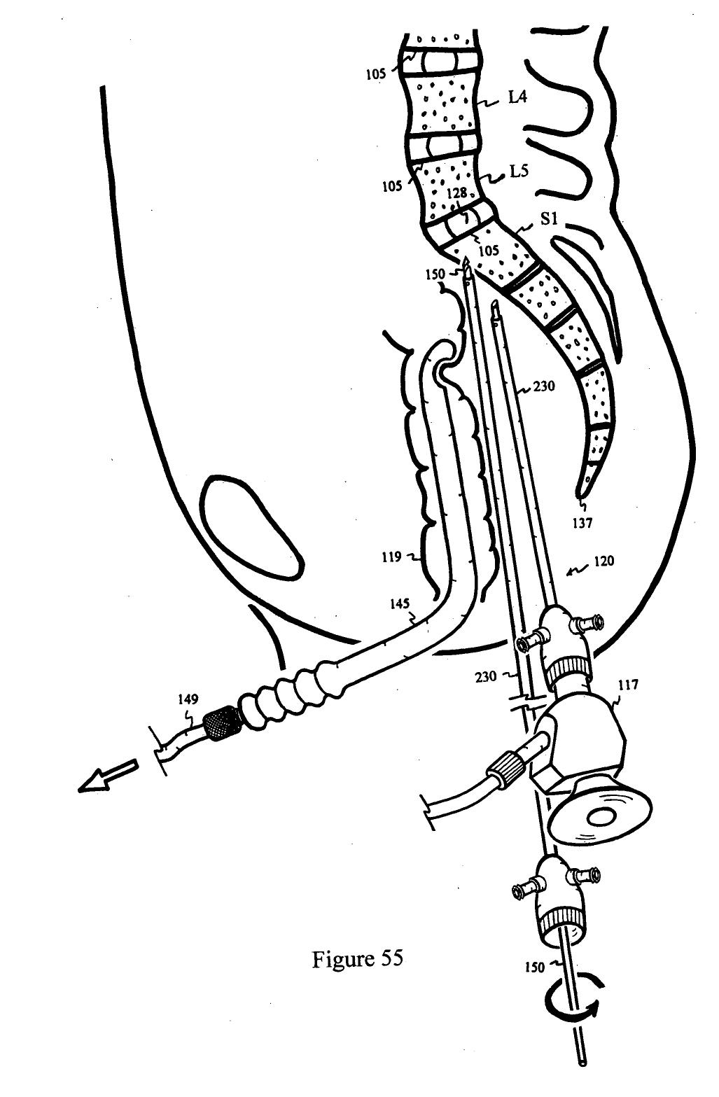

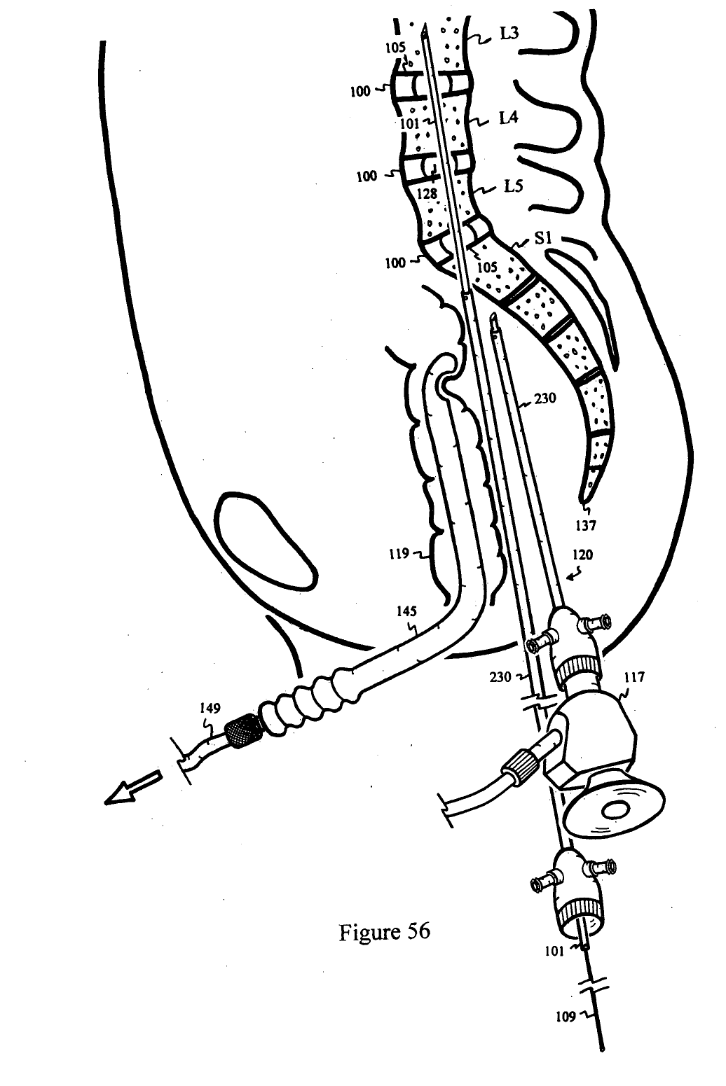

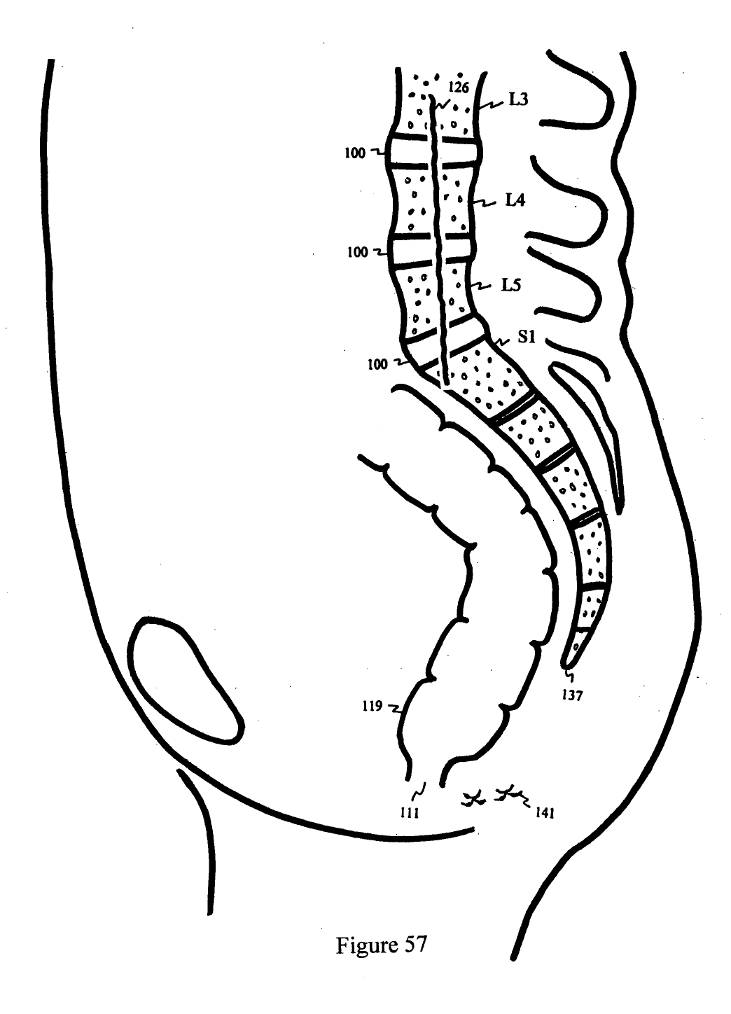

The colon 119 above the inferior fascia of pelvic diaphragm 120 blocks instruments from entering into the pelvic. The colon 119 is supple, compliant and stretchable. Hence, repositioning of the colon 119 for insertion of instruments, with a blunt rod 144 through the rectum 111 is difficult, as shown in FIG. 52. A colon positioner 145 contains a tubular body 147, a handle 148 connected to a vacuum line 149, a suction cup 146 at or near a blunt and curved distal end, as depicted in FIG. 53. A channel within the body 147 connects the suction cup 146 to the vacuum line 149. The suction cup 146 is located at the concave side of the distal curved portion of the positioner 145 for conforming to the direction and inner tissue of the colon 119. FIG. 54 shows the vacuum of the suction cup 146, holding the inner lining of the colon 119 and lifting the colon 119 to provide entry to the blunt obturators 141 within the sheaths 230. The obturators 141 advance with intermittent vacuum releases and advancements of the colon positioner 145. The obturators 141 are replaced with a drill 150 and an endoscope 117, as depicted in FIG. 55, drilling into vertebral bodies from S1 to possibly L3. The endoscope 117 is used to avoid puncturing of the median sacral artery and vein beneath the S1 vertebral body. The drill 150 is then replaced with a straight needle 101 containing a long conduit 126 abutting a plunger 109, as depicted in FIG. 56. The conduit 126 is deployed by withdrawing the needle 101 while holding the plunger 109 stationary. Hence, the conduit 126 re-establishes the exchange of nutrients and waste for multiple discs 100, as shown in FIG. 57.

It is generally accepted that disc 100 degeneration is largely related to nutritional and oxygen deficiency. In the supine position, disc pressure is low. Nutrients are drawn into the disc 100 through the semi-permeable conduit 126 to produce the water retaining sulfated glycosaminoglycans and increase the swelling pressure within the disc 100. Restoration of swelling pressure in the nucleus pulposus reinstates the tensile stresses within the collagen fibers of the annulus, thus reducing the inner bulging and shear stresses between annular layers. Similar to a re-inflated tire, disc 100 bulging is reduced and nerve impingement is minimized. The load on the facet joints 129 and segmental instability are reduced to ease wear and pain. Disc 100 height may increase to reverse spinal stenosis.

In daily activities, such as walking and lifting, pressure within the disc 100 greatly increases. The direction of the flow is then reversed within the conduit 126, flowing from high pressure within the disc 100 to low pressure within vertebral bodies 159. The lactic acid and carbon dioxide dissolved in the fluid within the nucleus pulposus is slowly expelled through the conduit 126 into the vertebral bodies 159, then to bodily circulation. As a result, the lactic acid concentration decreases, and pH within the disc 100 is normalized.

Furthermore, due to the continual supply of oxygen into the disc 100 through the conduit 126, lactic acid normally produced under anaerobic conditions may drastically decrease. Hence, the pain caused by acidic irritation to tissues, such as the posterior longitudinal ligament, superior 142 and inferior 143 articular processes of the facet joint 129, may quickly dissipate. Buffering agents, such as bicarbonate, carbonate or other, can be loaded or coated on the conduits 126 to neutralize lactic acid upon contact and spontaneously ease the pain.

Examples of conduit 126 material are included but are not limited to carboxymethyl cellulose, cellulose acetate, cellulose sulfate, cellulose triacetate, chitin, chitosan, chloroprene, ethylene-vinyl acetate, fluro-silicon hydrogel, hyaluronan, hyaluronate, neoprene, polyacrylamide, polyacrylate, polyacrylonitrile, poly-butylene terephthalate, poly-dimethyl-siloxane, poly-hydroxy-ethyl-acrylate, poly-hydroxy-ethyl-methacrylate, poly-hydroxy-methyl methacrylate, polymethacrylate, polymethylmethacrylate, polypropylene oxide, poly-siloxane, polyvinyl alcohol, poly-vinylpyrrolidone, silanol and vinyl methyl ether.

The endplate conduit 126 and the annular conduit 126 described in PCT/US2004/14368 (WO 2004/101015) may have different pore sizes to limit permeability. In addition, pore sizes may differ creating various permeabilities within sections of the conduit 126. The pore sizes of the conduit 126 may decrease toward the section near the nucleus pulposus 128 to minimize immune responses to the nucleus pulposus without excluding large nutrients from coming into or metabolites from going out of the middle portion of the annulus. Hence, the conduit 126 can have a permeable gradient from 200000, 100000, 70000, 50000, 30000, 10000, 5000, 3000, 1000 to 700 molecular weights of solutes. The pore sizes of the permeable gradient of the conduit 126 can range from 301 μm, 100 μm, 50 μm, 10 μm, 1 μm, 700 nm, 500 nm, 300 nm, 100 nm, 50 nm, 30 nm, 10 nm, 5 nm to 1 nm to prevent infiltration of IgA, IgD, IgE, IgG, IgM, cytokines or other initiators.

Excessive immune response to the conduit 126 and/or the nucleus pulposus 128 is often undesirable. Fibrous formation over the conduit 126 may affect the exchange of nutrients and waste between the disc 100 and bodily circulation. Exposure of the nucleus pulposus 128 may cause inflammation. Immuno inhibitor can be coated or incorporated into the conduit 126 to minimize fibrous formation or tissue response. Examples of immuno inhibitors include but are not limited to: aminopterin, azathioprine, chlorambucil, corticosteroids, crosslinked polyethylene glycol, cyclophosphamide, cyclosporin A, 6-mercaptopurine, methylprednisolone, methotrexate, niridazole, oxisuran, polyethylene glycol, prednisolone, prednisone, procarbazine, prostaglandin, prostaglandin E₁, steroids, other immune suppressant drug or other immune suppressant coating.

Hydrostatic pressure within the shunted disc 100 can be preserved by a swellable and semi-permeable coating over the conduit 126 to seal around the gap between the conduit 126 and annulus or between the conduit 126 and endplate 105. The swellable coating can be polyethylene glycol, crosslinked polyethylene glycol, polyurethane or other swellable material.

In addition, an initial supply of nutrients, such as magnesium trisilicate, magnesium mesotrisilicate, magnesium oxide, Magnosil, Pentimin, Trisomin, orthosilicic acid, magnesium trisilicate pentahydrate, Serpentine, sodium metasilicate, silanolates, silanol group, sialic acid, silicic acid, hydroxylysine, hydroxylproline, serine, threonine, boron, boric acid, glucose, glucuronic acid, galactose, galactosamine and/or glucosamine, can be used to coat the conduit 126 to enhance or initiate the production of sulfated glycosaminoglycans and collagen within the degenerative disc 100.

Healthy intervertebral discs 100 are avascular and immuno-isolated. To ensure the avascular and immuno-isolated conditions, conduits 126 can be incorporated, coated or partially coated with an anti-angiogenic compound. Examples of anti-angiogenic compounds are included but are not limited to Marimastat from British Biotech [a synthetic inhibitor of matrix metalloproteinases (MMPs)], Bay 12-9566 from Bayer (a synthetic inhibitor of tumor growth), AG3340 from Agouron (a synthetic MMP inhibitor), CGS 27023A from Novartis (a synthetic MMP inhibitor), COL-3 from Collagenex (a synthetic MMP inihibitor. Tetracycline® derivative), Neovastat from Aeterna, Sainte-Foy (a naturally occurring MMP inhibitor), BMS-275291 from Bristol-Myers Squib (a synthetic MMP inhibitor), TNP-470 from TAP Pharmaceuticals, (a synthetic analogue of fumagillin; inhibits endothelial cell growth), Thalidomide from Celgene (targets VEGF, bFGF), Squalamine from Magainin Pharmaceuticals (Extract from dogfish shark liver; inhibits sodium-hydrogen exchanger, NHE3), Combretastatin A-4 (CA4P) from Oxigene, (induction of apoptosis in proliferating endothelial cells), Endostatin collagen XVIII fragment from EntreMed (an inhibition of endothelial cells), Anti-VEGF Antibody from Genentech, [Monoclonal antibody to vascular endothelial growth factor (VEGF)], SU5416 from Sugen (blocks VEGF receptor signaling), SU6668 from Sugen (blocks VEGF, FGF, and EGF receptor signaling), PTK787/ZK 22584 from Novartis (blocks VEGF receptor signaling), Interferon-alpha from (inhibition of bFGF and VEGF production), Interferon-alpha from (inhibition of bFGF and VEGF production), EMD121974 from Merck KcgaA (small molecule blocker of integrin present on endothelial cell surface), CAI from NCI (inhibitor of calcium influx), Interleukin-12 from Genetics Institute (Up-regulation of interferon gamma and IP-10), IM862 from Cytran, Avastin, Celebrex, Erbitux, Herceptin, Iressa, Taxol, Velcade, TNP-470, CM101, Carboxyamido-triazole, Anti-neoplastic urinary protein, Isotretionin, Interferon-alpha, Tamoxifen, Tecogalan combrestatin, Squalamine, Cyclophosphamide, Angiostatin, Platelet factor-4, Anginex, Eponemycin, Epoxomicin, Epoxy-β-aminoketone, Antiangiogenic antithrombin III, Canstatin, Cartilage-derived inhibitor, CD59 complement fragment, Fibronectin fragment, Gro-beta, Heparinases, heparin hexasaccharide fragment, Human chorinonic gonadotropin, Interferon (alpha, beta or gamma), Interferon inducible protein (IP-10), Interleukin-12 (IL-12), Kringle 5 (plasminogen fragment), Tissue inhibitors of metalloproteinases, 2-Methoxyestradiol (Panzem), Placental ribonuclease inhibitor, Plasminogen activator inhibitor, Prolactin 16 kD fragment, Retinoids, Tetrahydrocortisol-S, Thrombospondin-1, Transforming growth factor beta, Vasculostatin, and Vasostatin (calreticulin fragment).

It is to be understood that the present invention is by no means limited to the particular constructions disclosed herein and/or shown in the drawings, but also includes any other modification, changes or equivalents within the scope of the claims. Many features have been listed with particular configurations, curvatures, options, and embodiments. Any one or more of the features described may be added to or combined with any of the other embodiments or other standard devices to create alternate combinations and embodiments. The elastically curved needle 101 can be called the resilient needle 101. The rigid needle 220, needle 101 or drill sleeve 313 can be generally described in the claims as a sheath with a lumen. The vertebral body 159 can be called vertebrae.

It should be clear to one skilled in the art that the current embodiments, materials, constructions, methods, tissues or incision sites are not the only uses for which the invention may be used. Different materials, constructions, methods, coating or designs for the conduit 126 can be substituted and used. Nothing in the preceding description should be taken to limit the scope of the present invention. The full scope of the invention is to be determined by the appended claims.

1. A conduit for re-establishing exchange of nutrients and waste between an intervertebral disc and bodily circulation, the conduit comprising:
an elongated member having a first end and a second end and formed of a biocompatible material, said elongated member being locatable such that a first portion of said elongated member is within a patient's intervertebral disc,
and a plug located around said elongated member between said first and second ends, said plug having a tapered portion.

2. The conduit of claim 1, wherein said plug has a longitudinal slit through a side thereof.

3. The conduit of claim 2, wherein said slit extends from a first end to a second end of said plug, thereby allowing said plug to be compressed to a smaller diameter.

4. The conduit of claim 1, wherein said plug has at least one ridge extending from an outside surface thereof.

5. The conduit of claim 4, wherein said at least one ridge forms threading around a periphery of said plug.

6. The conduit of claim 2, wherein a first end of said plug forms a nut, said first end of said plug adjacent a wider end of said tapered portion.

7. The conduit of claim 6, wherein said slit extends through said nut, thereby allowing said nut to deform when said plug is deformed by being forced into an opening.

8. The conduit of claim 6, further comprising a sleeve having an internal opening sized and configured to mate with said nut, such that when said internal opening is located around said nut and said sleeve is twisted, said plug will turn.

9. The conduit of claim 1, wherein a first end of said plug forms a nut, said first end of said plug adjacent a wider end of said tapered portion.

10. The conduit of claim 1, further comprising a coating, said coating selected to inhibit immune response.

11. The conduit of claim 10, wherein said coating is selected from the group of coating including: aminopterin, azathioprine, chlorambucil, corticosteroids, crosslinked polyethylene glycol, cyclophosphamide, cyclosporin A, 6-mercaptopurine, methylprednisolone, methotrexate, niridazole, oxisuran, polyethylene glycol, prednisolone, prednisone, procarbazine, prostaglandin, prostaglandin E₁, steroids, other immune suppressant drug and other immune suppressant coating.

12. The conduit of claim 1, further comprising a swellable coating, said swellable coating selected to preserve hydrostatic pressure within said intervertebral disc.

13. The conduit of claim 12, wherein said swellable coating is selected from the group of coating including: polyethylene glycol, crosslinked polyethylene glycol, polyurethane and other swellable material.

14. The conduit of claim 1, further comprising an initial supply of nutrients to enhance production of sulfated glycosaminoglycans and collagen.

15. The conduit of claim 14 wherein said initial supply of nutrients is selected from the group of nutrients including: magnesium trisilicate, magnesium mesotrisilicate, magnesium oxide, Magnosil, Pentimin, Trisomin, orthosilicic acid, magnesium trisilicate pentahydrate, Serpentine, sodium metasilicate, silanolates, silanol group, sialic acid, silicic acid, hydroxylysine, hydroxylproline, serine, threonine, boron, boric acid, glucose, glucuronic acid, galactose, galactosamine and glucosamine.

16. The conduit of claim 14, wherein said initial supply of nutrients coats said conduit.

17. The conduit of claim 14, wherein said initial supply of nutrients is within said conduit.

18. The conduit of claim 1, further comprising a coating, said coating selected to inhibit blood vessel ingrowth.

19. The conduit of claim 18, wherein said coating is selected from the group of coatings consisting of: Marimastat, Bay 12-9566, AG3340, CGS 27023A, COL-3, Tetracycline®, Neovastat, Sainte-Foy, BMS-275291, TNP-470, Thalidomide, Squalamine, Combretastatin A-4, Endostatin collagen XVIII fragment, Anti-VEGF Antibody, SU5416, SU6668, PTK787/ZK 22584, Interferon-alpha, Interferon-alpha, EMD121974, CAI, Interleukin-12, IM862, Avastin, Celebrex, Erbitux, Herceptin, Iressa, Taxol, Velcade, TNP-470, CM101, Carboxyamido-triazole, Anti-neoplastic urinary protein, Isotretionin, Interferon-alpha, Tamoxifen, Tecogalan combrestatin, Squalamine, Cyclophosphamide, Angiostatin, Platelet factor-4, Anginex, Eponemycin, Epoxomicin, Epoxy-β-aminoketone, Antiangiogenic antithrombin III, Canstatin, Cartilage-derived inhibitor, CD59 complement fragment, Fibronectin fragment, Gro-beta, Heparinases, heparin hexasaccharide fragment, Human chorinonic gonadotropin, Interferon (alpha, beta or gamma), Interferon inducible protein (IP-10), Interleukin-12 (IL-12), Kringle 5 (plasminogen fragment), Tissue inhibitors of metalloproteinases, 2-Methoxyestradiol (Panzem), Placental ribonuclease inhibitor, Plasminogen activator inhibitor, Prolactin 16 kD fragment, Retinoids, Tetrahydrocortisol-S, Thrombospondin-1, Transforming growth factor beta, Vasculostatin, and Vasostatin (calreticulin fragment).

20. The conduit of claim 1, wherein said biocompatible material is selected from the group of materials consisting of: carboxymethyl cellulose, cellulose acetate, cellulose sulfate, cellulose triacetate, chitin, chitosan, chloroprene, ethylene-vinyl acetate, fluro-silicon hydrogel, hyaluronan, hyaluronate, neoprene, polyacrylamide, polyacrylate, polyacrylonitrile, poly-butylene terephthalate, poly-dimethyl-siloxane, poly-hydroxy-ethyl-acrylate, poly-hydroxy-ethyl-methacrylate, poly-hydroxy-methyl methacrylate, polymethacrylate, polymethylmethacrylate, polypropylene oxide, poly-siloxane, polyvinyl alcohol, poly-vinylpyrrolidone, silanol and vinyl methyl ether.

21. A conduit for re-establishing exchange of nutrients and waste between an intervertebral disc and bodily circulation, the conduit comprising:
an elongated member having a first end and a second end and formed of a permeable, biocompatible material, said elongated member being locatable such that a first portion of said elongated member is within a patient's intervertebral disc, wherein a permeability of said biocompatible material varies along the length of said conduit.

22. The conduit of claim 21, wherein a pore size of said conduit varies along a length of said conduit.

23. The conduit of claim 22, wherein said pore size decreases toward said first portion.

24. The conduit of claim 22, wherein said pore size is larger than 300 μm.

25. The conduit of claim 22, wherein said pore size is smaller than 2 nm.

26. A method for re-establishing an exchange of nutrients and waste between an intervertebral disc and bodily circulation, the method comprising the steps of:
(a) inserting a needle of a delivery device through a vertebrae into the intervertebral disc;
(b) actuating the delivery device to deploy a conduit; and
(c) removing said needle from the vertebrae.

27. The method of claim 26, wherein said needle extends through an endplate.

28. The method of claim 26, wherein said needle extends through a pedicle of the vertebrae into an endplate.

29. The method of claim 26, wherein said needle extends through an anterior face of the vertebrae into an endplate.

30. The method of claim 26, wherein a drill is used to create an opening through the vertebrae.

31. The method of claim 26, wherein said needle extending through more than one vertebrae and more than one endplate.
